(19) United States
(12) Patent Application Publication
(10) Pub. No.: US 2020/0401465 A1
(43) Pub. Date: Dec. 24, 2020

(54) APPARATUSES, SYSTEMS, AND METHODS FOR PROVIDING HEALTHCARE INTEGRATIONS

(71) Applicant: THE BRIDGECR LLC, Nashville, TN (US)

(72) Inventors: Joshua Adam Douglas, Nashville, TN (US); Matthew Thomas Wimberley, Coden, AL (US); Clinton Thomas Johnson, Nashville, TN (US)

(21) Appl. No.: 16/907,271

(22) Filed: Jun. 21, 2020

Related U.S. Application Data
(60) Provisional application No. 62/864,768, filed on Jun. 21, 2019.

(51) Int. Cl. G06F 9/54; G16H 80/00; G16H 40/00

(52) U.S. Cl. CPC G06F 9/541; G16H 40/00; G16H 80/00

(57) ABSTRACT

Systems, methods, and apparatuses for providing healthcare integrations, are provided. The system may include a network, a server communicatively coupleable to the network, the server configured to generate an interface accessible via the network, a plurality of third-party service Application Programming Interfaces (APIs) coupleable to the network, and a user device coupleable to the network, the user device configured to access the interface generated by the server to generate a connector, the connector configured to include at least one of the plurality of third-party service APIs.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application claims the benefit of U.S. Provisional Patent No. 62/864,768, titled “Apparatuses, Systems, and Methods for Providing Healthcare Integrations,” filed Jun. 21, 2019, which is hereby incorporated by reference in its entirety.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present disclosure relates generally to apparatuses, systems, and methods for providing healthcare integrations.

Healthcare systems and corresponding data storage systems often vary wildly, both in physical and virtual configuration. Many systems are unable to communicate with one another, instead providing disparate interfaces and access methods.

Currently, the market tends to approach the problem by implementing one-off custom software in which developers code to create “adapters” or communication configurations. These adapters are the source code that contains details for connecting to a healthcare system. This current approach to accessing healthcare systems requires extensive custom programming and technical knowledge (such as information regarding how to connect, details of repositories, etc.), and often relies on costly niche programming. In addition, the process of creating adapters can take months to complete for just one integration and can be cost prohibitive to parts of the market.

Accordingly, what is needed is a way to connect to healthcare systems which does not require one-off customized software adapters but provides access to such healthcare systems.

### BRIEF SUMMARY

Embodiments of the present disclosure provide apparatuses, systems, and methods for providing healthcare integrations.

The Bridge Connector healthcare integration ecosystem provides a unique and flexible solution to the problem of data interoperability. Solutions consistent with the present disclosure provide the ability to connect disparate systems together, thereby allowing for an extremely efficient and intuitive way to create complex integrations without the need for code or even technical knowledge. This may be accomplished in various embodiments, for example, by providing an intuitive user interface (UI) presented to a user which allows the user to create an integration, workflow, and/or workflow step without requiring custom coding. Artificial Intelligence (AI) may be used to assist in creating integrations consistent with the present disclosure.

Unlike existing middleware solutions which require custom coding to parse into a particular data model, implementations consistent with the present disclosure enable dynamic and responsive integration and workflow creation and modification.

According to aspects of the present disclosure, provided is a method for providing healthcare integrations. The method includes receiving a request to generate a connector, identifying a source and a destination for the connector, selecting at least one action associated with the connector, defining a parameter associated with the at least one action, and generating the connector based at least in part upon the identified source and destination, and the selected at least one action, the defined parameter.

According to a further aspect of the present disclosure, provided is an apparatus for providing healthcare integrations. The apparatus includes a core gateway configured to communicate with at least one third-party Application Programming Interface (API), a storage configured to store connector profile information, a server configured to generate an interface associated with at least one connector, the interface configured to permit a user to generate at least one connector, an analysis engine configured to interpret at least one communication associated with a connector and configured to store a representation of the interpreted at least one communication associated with the connector, and a connector generator, the connector generator configured to generate a connector based at least in part upon.

According to a still further aspect of the present disclosure, provided is a system for providing healthcare integrations. The system includes a network, a server communicatively coupleable to the network, the server configured to generate an interface accessible via the network, a plurality of third-party service Application Programming Interfaces (APIs) coupleable to the network, and a user device coupleable to the network, the user device configured to access the interface generated by the server to generate a connector, the connector configured to include at least one of the plurality of third-party service APIs.

Numerous other objects, features, and advantages of the present disclosure will be readily apparent to those skilled in the art upon a reading of the following disclosure when taken in conjunction with the accompanying drawings.

### BRIEF DESCRIPTION OF THE SEVERAL VIEWS OF THE DRAWINGS

FIG. 1 illustrates an exemplary embodiment of a functional network diagram of a system according to aspects of the present disclosure.

FIG. 2 illustrates an exemplary embodiment of a partial functional block diagram according to aspects of the present disclosure.

FIG. 3 illustrates an exemplary embodiment of a process flow and functional network diagram consistent with the present disclosure

FIG. 4 illustrates an exemplary embodiment of a partial block diagram of a flow of data over multiple methods/protocols according to aspects of the present disclosure.

FIG. 5 illustrates an exemplary embodiment of a process for selectively binding a connector profile according to aspects of the present disclosure.

FIG. 6 illustrates an exemplary embodiment of user interaction with an interface and corresponding operations according to aspects of the present disclosure (e.g., as a workflow creation user flow diagram).

FIG. 7 illustrates an exemplary embodiment of a dashboard interface according to aspects of the present disclosure.

FIG. 8 illustrates an exemplary embodiment of an interface according to aspects of the present disclosure.

FIG. 9 illustrates an exemplary embodiment of a partial view of an interface for a user to bridge two or more Vendors/apps according to aspects of the present disclosure.

FIG. 10 illustrates an exemplary interface for manual bridge creation according to aspects of the present disclosure (e.g., as automated at least in part from a template in various embodiments).

FIG. 11 illustrates an exemplary interface 1100 associated with an action step in the workflow of FIG. 10 (e.g., as optionally generated at least in part according to template details) according to aspects of the present disclosure.

FIG. 12 illustrates an exemplary embodiment of a workflow creation interface according to aspects of the present disclosure.

FIG. 13 illustrates an exemplary embodiment of a workflow creation interface according to aspects of the present disclosure.

FIG. 14 illustrates an exemplary embodiment of a user interface identifying one or more sub accounts associated with a user according to aspects of the present disclosure.

FIG. 15 illustrates an exemplary embodiment of a workflow step setup interface presented to a user according to aspects of the present disclosure.

FIGS. 16 and 16A-D collectively illustrate an exemplary embodiment of a connector creation user flow according to aspects of the present disclosure (e.g., as a connector creation user flow diagram) according to aspects of the present disclosure.

FIG. 17 illustrates an exemplary embodiment of an exemplary administration interface in accordance with the present disclosure.

FIG. 18 illustrates an exemplary implementation of workflow processing according to aspects of the present disclosure.

FIG. 19 illustrates an exemplary embodiment of a partial block network diagram according to aspects of the present disclosure.

FIG. 20 illustrates an exemplary embodiment of a partial block diagram of a system according to aspects of the present disclosure.

FIG. 21 illustrates an exemplary embodiment of an interface for creating or editing a connector according to aspects of the present disclosure.

FIG. 22 illustrates an exemplary embodiment of an interface for viewing or specifying one or more actions for a connector according to aspects of the present disclosure.

FIG. 23 illustrates an exemplary embodiment of an output schema interface according to aspects of the present disclosure.

FIG. 24 illustrates an exemplary embodiment of a complete version of an interface of FIG. 21 according to aspects of the present disclosure.

FIG. 25 illustrates an exemplary embodiment of an interface of FIG. 22 according to aspects of the present disclosure.

FIG. 26 illustrates an exemplary embodiment of an interface for creating an authentication flow for a connector according to aspects of the present disclosure.

FIG. 27 illustrates an exemplary embodiment of a partial interface for a second step of the interface of FIG. 26 according to aspects of the present disclosure.

FIG. 28 illustrates an exemplary embodiment of an SFTP configuration interface according to aspects of the present disclosure.

FIG. 29 illustrates an exemplary embodiment of an interface for viewing or specifying one or more actions for a connector according to aspects of the present disclosure.

FIG. 30 illustrates an exemplary embodiment of an interface element for selecting a step action according to aspects of the present disclosure.

FIG. 31 illustrates an exemplary embodiment of a partial block diagram of a destinations entity.

FIG. 32 illustrates an exemplary embodiment of a simplified block diagram reflecting an alternative embodiment corresponding to FIG. 3 according to aspects of the present disclosure.

### DETAILED DESCRIPTION

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the disclosure and do not delimit the scope of the invention.

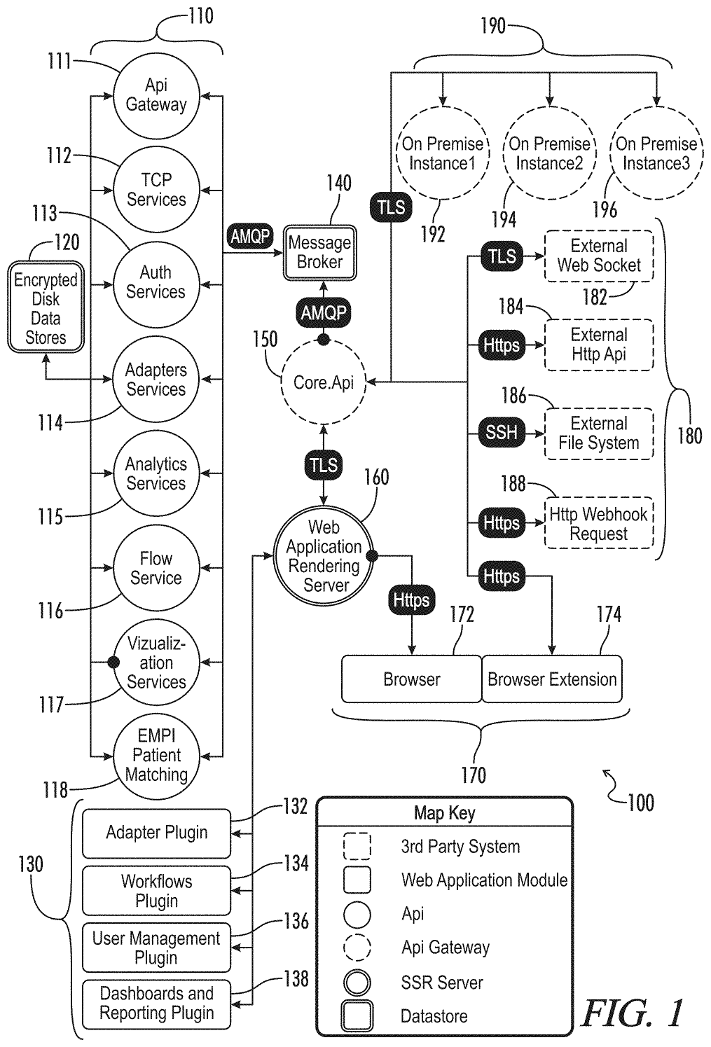

Referring generally to FIGS. 1-32, various exemplary apparatuses, systems, and associated methods according to the present disclosure are described in detail. Where the various figures may describe embodiments sharing various common elements and features with other embodiments, similar elements and features are given the same reference numerals and redundant description thereof may be omitted below.

Various embodiments of an apparatus according to the present disclosure may provide healthcare integrations.

As illustrated, for example, at least by FIG. 1, a system 100 consistent with the present disclosure may include at least one of an application programming interface (API) or server layer (collectively referred to herein as a core Application Programming Interface (API) 150 and/or core.API 150), a network or gateway layer, and/or a server-side client layer. The core API 150 may be configured to handle all or a part of communication(s) to/from an external device. The core API 150 may be configured to leverage a message queue where one more microservices listen on for further processing, for example as a message broker 140. The core API 150 may variously rely upon one or more connector microservices (e.g., service(s) 110) to set up and/or maintain communication for one or more workflow steps. Each workflow step may leverage a connector profile (configuration) from the connectors microservice, the connector configuration optionally providing instructions to handle the payload from the gateway. Thus, the process in one exemplary embodiment may rely upon four different microservices to process and route a message: connectors, workflows, auth(authentication and authorization, for example via auth services 113), and the core API 150. One or more microservices (e.g., service(s) 110) consistent with the present disclosure may include or comprise one or more of an API gateway 111, TCP service(s) 112, Auth service(s) 113, Connector service(s) 114, analytics service(s) 115, flow service(s) 116), visualization service(s) 117, and/or EMPI service(s) 118.

One or more workflow integration microservices may be configured to permit users of the core application to create one or more workflows comprised of sequential steps. Each step may correspond to an action to be taken using a connector profile from one or more connector microservice(s) (e.g., sources of information). Each step may optionally provide results to a next or subsequent step for further chaining. A third-party API may communicate with the core API 150 via HTTP/REST messaging in an exemplary embodiment. The core API 150 may be coupled to a database, such as a relational database. The core API 150 may be communicatively coupled to a data structure store configured to provide at least one of a database, a cache, and/or a message broker 140. In various embodiments, the data structure store may be an in-memory data store component or system. A third-party service may communicate with the core API 150 over any protocol, including polling HTTP RESTful services, Opening and managing configurable IP-SEC tunnels over TCP, configurable webhooks over HTTP, Web Socket Secure over TCP, secure file transfer protocols such as SFTP, and S3 buckets (e.g., via the TCP services 112). The core API 150 microservices leverage relational, document, graph, in memory, and block chain-based data stores to provide the aforementioned functionality. In various embodiments, the data structure store may be an in-memory data store component or system.

The analytics engine (e.g., corresponding to analytics services 115) of the core API 150 may be configured to provide one or more of logging, data analysis, and/or prediction to provide a data-driven approach to creating integrations with configurable compliance. In various embodiments, the analytics engine may be configured to predict one or more parameters of an integration using artificial intelligence (AI), for example, based at least in part upon knowledge gained from previous operations, an expected modification of data, a predicted communication setting, or any other predicted or predictable variable or set of information. A chart and report rendering microservice module of the core API 150 may be configured to provide flexible data tools, such as charting, that may be used to power dashboards and/or operations. The core API 150 may also include an Extensible and Scalable Master Patient Index (EMPI) (e.g., via the EMPI Patient matching 118) which provides a configurable patient matching microservice and may include an SDK configured to leverage OpenEMPI to provide flexible patient matching in one or more workflow steps.

One or more plugins may be implemented, for example at least one of a connector plugin 132, a workflow plugin, a user management plugin 136, and/or a dashboard and reporting plugin 138 may be communicatively coupleable to a web application rendering server 160. The web application rendering server 160 may be coupleable to the Core API 150, for example via a Transport Level Security (TLS) connection. The message broker 140 may be communicatively coupleable to the core API 150 via at least one Advanced Message Queuing Protocol (AMQP) connection, via wired connection, wireless connection, or combination thereof. Similarly, at least one of the services 110 may be communicatively coupleable to the message broker 140 via at least one AMQP connection, via wired connection, wireless connection, or combination thereof.

As illustrated, for example by FIG. 1, an in-memory data store component or system may be communicatively coupled to a data cache and a core network. The core API 150 may leverage a mesh network design pattern allowing it to be scaled and deployed to on-premise solutions (e.g., on-premise solutions 190, such as on-premise instance1 192, on-premise instance2 194, on-premise instance3 196, etc.). In that case, many nodes of the gateway are managed and represented by the connectors microservice, each one is identified by its own connector profile which can then be attached to a workflow step and utilized. The gateway node may include one or more configurable instances to permit real-time data communication between the network and a facility's on-site instance represented and managed by a connector profile, thereby allowing systems without external facing communication to attach one or many on premise nodes to workflow steps for communication with on-site instances. Accordingly, it is possible to provide a way for facilities without requiring an API to integrate with the Bridge Connector platform.

The core client may include one or more of a connector creation/management wizard, a workflow creation/management wizard, and/or a monitoring/reporting module. The browser 170, 172 may include a browser extension 174 configured to communicate with the core network. The browser may be further configured to send information to and receive information from the core client, for example via an HTTPS connection to the web application rendering server 160. The browser extension 174 and connector automation tool may permit tracking communication of a client's healthcare services and may relay at least a portion of that information back to a service associated with the workflow(s) to be parsed for use as or in conjunction with a connector. In various embodiments, the browser extension 174 may be configured to either actively or passively capture or view information exchange and/or to generate one or more connectors based at least in part upon the captured or viewed information.

One or more messages exchanged may be formatted according to one or more protocols, including Health Level 7 (HL7), Extensible Markup Language (XML), JavaScript Object Notation (JSON), or any other format readable and/or operable according to the present disclosure. A user of the systems and apparatuses described herein may include one or more users associated with a healthcare entity such as a hospital, laboratory, agency, doctor's office, or the like. Although described with reference to healthcare, it should be appreciated that one or more aspects of the present disclosure may be implementable in other fields without departing from the spirit and scope of the present disclosure, for example the legal field, business field, or any other field in which information is exchanged and/or confidentiality of exchanged information is necessary or optional.

During operation, a user may be provided with a graphical user interface (GUI) useable to provide one or more connector microservice(s) and/or workflow steps for information exchange. Although described as a connector, it should be appreciated that a connector may be a generic high-level detail for communication with a service, and a connector profile may include instance-level detail information pertaining to a specific customer or account. In one embodiment, the user may select an information source such as a medical record provider and may optionally select one or more retrieval parameters and/or workflow steps. The system is configured to receive the user's input, to determine whether an integration exists corresponding to the user's input, and to optionally generate an automation if such integration does not exist or is not currently operational. The system optionally views and/or captures real-time use data to create and/or modify integrations, for example by creating a new integration or modifying an operational parameter of an existing integration based upon the viewed and/or captured information associated with a particular user or otherwise obtained by the system (e.g., from other users or interactions). Systems consistent with the present disclosure are further capable of providing smart connector microservice(s) for integrations, meaning the system may provide real-time determination of needs or statuses or at least one of a sender or receiver.

The core application is configured in various embodiments to handle connector and connector profile information. The core application may be further configured to handle workflow orchestration and/or analytics. The core application may be implemented as a plurality of microservices in one or more embodiments. The core network may provide internet-based web connectivity. The core network may be configured in various embodiments to transmit and/or receive messages formatted according to a predetermined format such as HL7 in various embodiments. The core network may be further configured to listen for and/or perform at least one operation in association with a particular message or messages having a particular format or formatting.

The Bridge Connector, the core network, the core client, and the browser extension may include one or more components, modules, and/or protocols to perform one or more operations discussed herein. Notwithstanding the above components, it should be appreciated that one or more additional, fewer, or alternative protocols and/or components may be used without departing from the spirit and scope of the present disclosure.

One or more computing components and/or functional operations described herein may be connected to or otherwise accessible via a network. In one exemplary embodiment, the network includes the Internet, a public network, a private network, or any other communications medium capable of conveying electronic communications. Connection between elements or components is configured to be performed by wired interface, wireless interface, or a combination thereof, without departing from the spirit and the scope of the present disclosure. In one exemplary operation, at least one computing component and/or functional element is configured to store one or more sets of instructions in a storage element. The one or more sets of instructions may be configured to be executed by a microprocessor to perform operations corresponding to the one or more sets of instructions.

In various exemplary embodiments, at least one computing element is implemented as at least one of a desktop computer, a server computer, a laptop computer, a smart phone, or any other electronic device capable of executing instructions. The microprocessor may be a generic hardware processor, a special-purpose hardware processor, or a combination thereof. In embodiments having a generic hardware processor (e.g., as a central processing unit (CPU) available from manufacturers such as Intel and AMD), the generic hardware processor is configured to be converted to a special-purpose processor by means of being programmed to execute and/or by executing a particular algorithm in the manner discussed herein for providing a specific operation or result.

One or more computing component and/or functional element may be configured to operate remotely and may be further configured to obtain or otherwise operate upon one or more instructions stored physically remote from the computing component and/or functional element (e.g., via client-server communications and/or cloud-based computing).

At least one computing component may include a display unit. The display unit may be embodied within the computing component or functional element in one embodiment and may be configured to be either wired to or wirelessly interfaced with at least one other computing component or functional element. The display unit may be configured to operate, at least in part, based upon one or more operations of the described herein, as executed by the microprocessor.

In an exemplary embodiment of a flow of data over multiple methods/protocols, once data is received on the core API 150, the gateway authenticates and authorizes the message over WSS then retrieves the connector profile information over WSS, from there this payload is queued. The workflow microservice retrieves the message from the queue and processes the step, optionally making the results available to any next step created by the user. The process begins when a request is received, for example an HTTP request. The request is provided to at least one of a connector manager and/or configuration manager associated with a system or interface such as a PenPalHC system or interface. The system then provides a connector configuration request to a database containing at least one connector configuration. In one exemplary embodiment, the database contains a plurality of connector configuration information. A connector manager provides a request with profile information to a response factory. The response factory may include one or more connectors, such as a Json API connector, an XML API connector, a SOAP API connector, or any other connector. One or more of the connectors may include signer and validator modules. One or more of the connectors of the response factory may execute the request, for example, my transmitting information to a third-party application and/or receiving information from the third-party application and optionally returning that information to a sender of the original request or entity associated with such sender.

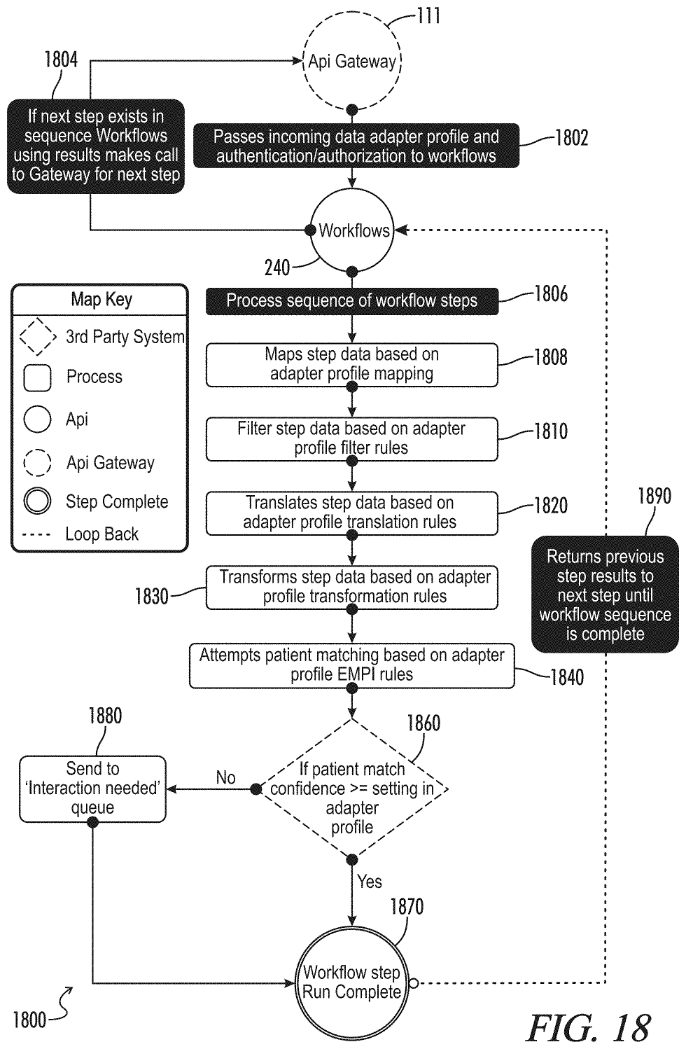

An exemplary process flow (e.g., as described herein with reference to FIG. 18) may include how RESTful messages are handled dynamically, for example when a payload gets to workflow first mapping the step data based on the connector profile mapping, next filtering the data based on the connector profile filter rules, then translating and performing any transformation(s) set by the connector profile, next patient matching logic is processed if EMPI rules exist on the connector profile, finally the result is provided to the next step. The process begins by fetching configuration data (or otherwise receiving such data) from connector microservice(s). It is determined whether data exist corresponding to the received configuration data. If data does not exist, the process terminates. If data does exist, the process continues to a step of providing configuration data to a configuration manager for binding. The process then passes configuration data to a response manager. The response manager then signs the request and validates it. It is determined whether the request is signed and validated. If the request is not signed and validated, the process terminates. If, however, the request is signed and validated, the process continues by sending a request to a third-party application. A return response from the third-party application is provided to the application core and the process then ends.

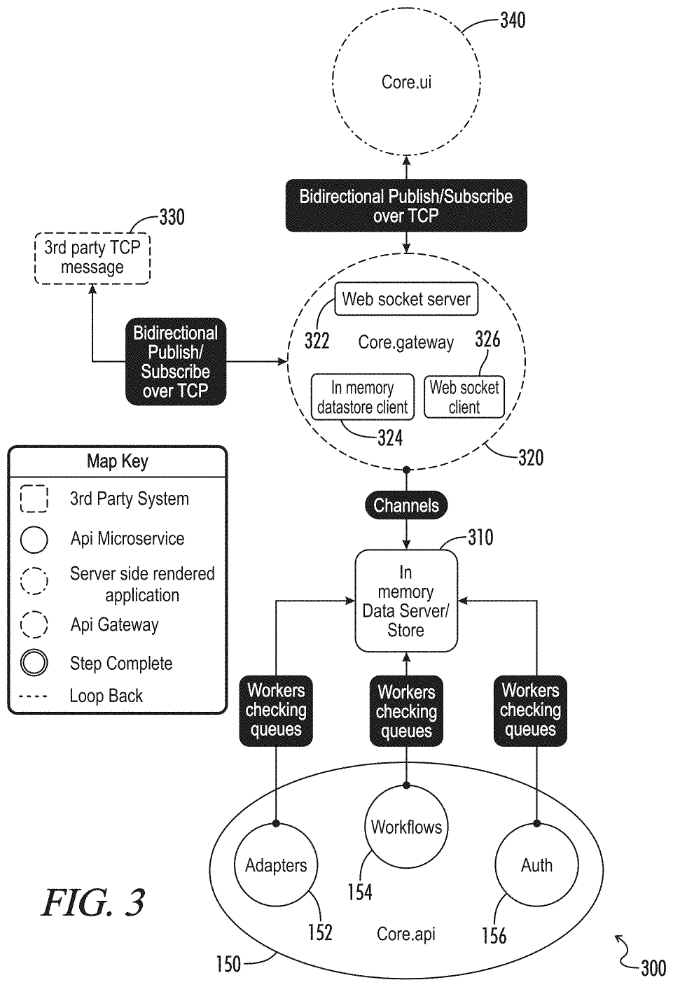

FIG. 3 illustrates an exemplary embodiment of a process flow and functional network diagram consistent with the present disclosure. As illustrated, a command may be received and at least one set of information extracted therefrom. An integration, workflow step, and/or parameter associated therewith may be loaded and/or transformed based least in part upon the extracted information. Information may then be exchanged and/or tracked in accordance with the previous description herein.

In an exemplary embodiment also consistent with FIG. 3, a relational database may be coupled to the core API 150. In embodiments consistent with FIG. 3, the in-memory data store may be coupled to the core API 150 microservice, for example over TCP by way of the web socket server in the gateway (e.g., via the TCP service 112). The gateway may then use its web socket client to communicate across channels to the core API 150 which as one or more worker jobs listening. During operation, the core API 150 may be further coupled to at least one worker in communication with an in-memory data store server or service. The in-memory data store server may include or otherwise be coupleable to a data cache. The in-memory data store server or service may be coupled to a node via one or more communication channels. The node includes an in-memory data store client a web socket client and/or a web socket server. One or more third-party healthcare messages may be published and/or subscribed via the web socket server/client of the node. The node may be configured to exchange server-side rendering information in relation to the core client.

Systems in accordance with the present disclosure may permit users to create connectors via an application (e.g., a web application) which provides an automated API document upload/parsing service, a data transformation/translation service, and/or a connector and data mapping prediction server to enable a quick and user-intuitive process.

FIG. 3 illustrates an exemplary embodiment of a functional network diagram in accordance with the present disclosure. The system includes a core client, a core network, and a core server. The core client includes at least one web socket client, at least one browser coupled to the web socket client(s), and optionally includes at least one third-party application coupled to a web socket client. One or more web socket clients is coupleable to a channel of the core network. The web socket client is configured to transmit and/or receive at least one set of data to/from the web socket server of the core network. The web socket server is configured to communicate with an in-memory data store client via at least one middleware of the core network. The in-memory data store client of the core network is configured to communicate with an in-memory data store client of the core server via at least one channel. The in-memory data store client of the core server is configured to communicate with at least one worker of the core server. The at least one worker is configured to communicate with at least one queue. One or more queues is configured to receive at least one event from at least one subscriber. Additionally or alternatively, one or more queues is configured to receive at least one job (either scheduled or unscheduled) from at least one subscriber.

Apparatuses, systems, and methods consistent with the present disclosure may be configured to provide a secure link between one or more connected systems. The secure link may include one or more of At-Rest and/or In-Transit encryption, IPSEC security, TLS security, TDE security, HIPAA compliance standards, and/or may comply with NIST 800-53 security standards.

Additional aspects of the present disclosure are provided in the attached Figures. FIG. 1 includes an exemplary configuration of systems, apparatuses, and methods consistent herewith (e.g., as an ecosystem-level diagram). An API gateway 111 may be coupled to one or more of an in-memory data store, a web application rendering server, one or more managed node of the gateway instances, and/or one or more third-party communication interfaces (e.g., third party TCP socket(s), HTTP API(s), file system(s), and/or webhook request(s). In an exemplary embodiment, at least one managed node of the gateway interface may be an on-premise communication node. The web application rendering server may be further coupled to one or more of a browser, a browser extension, a connector microservice(s) connector module, a workflows module, a user management module, and/or one or more dashboards and reporting interfaces or systems. The in-memory data store may be coupled to one or more interfaces, including an Authentication and authorization microservice API, a connector microservice(s) API, an analytics engine API, a workflows API, a chart and report rendering microservice or module, and/or an EMPI patient mapping interface or module. One or more of the Authentication and authorization microservice API, a connector microservice(s) API, an analytics engine API, a workflows API, a chart and report rendering microservice or module, and/or an EMPI patient mapping interface or module may be further coupled to one or more disk-based data stores (e.g., encrypted disk data stores 120 via the connector service 114).

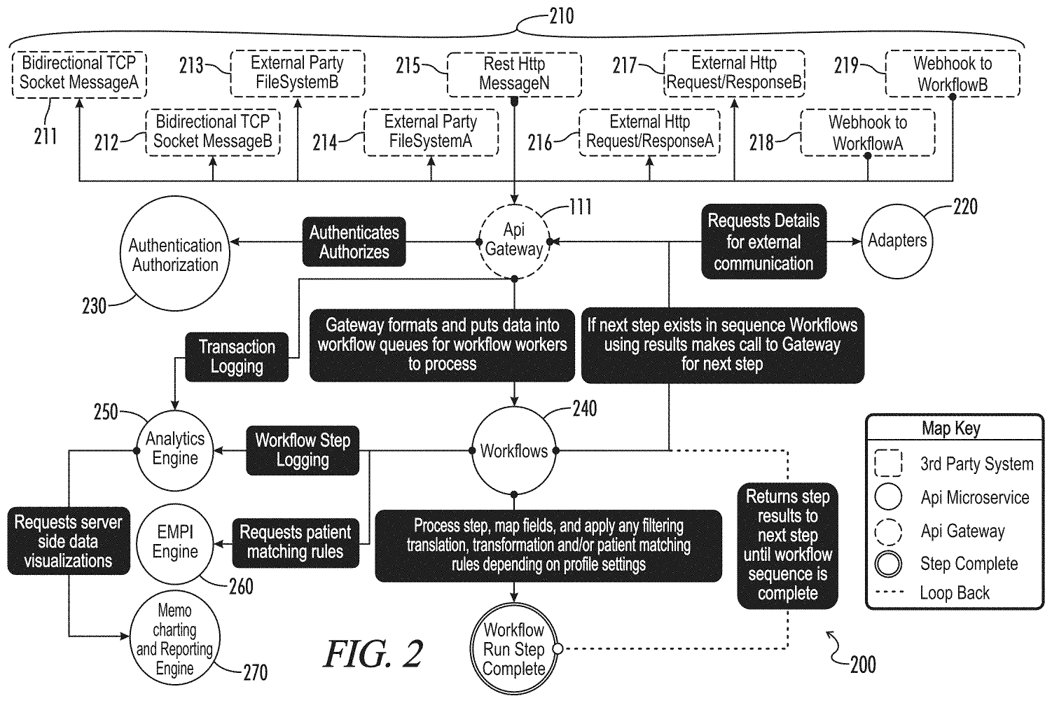

FIG. 2 illustrates an exemplary functional diagram according to aspects of the present disclosure (e.g., as a dataflow model). The API gateway 111 is functionally coupleable to an authentication and authorization microservice module 230 configured to store and/or receive at least a portion of information used for authentication of a user, data source, and/or data recipient, or entity associated therewith. The API gateway 111 is further configured be communicatively coupleable with one or more third-party system interfaces 210, for example via a bidirectional TCP socket message A 211, a Bidirectional TCP socket message B 212, an external party file system A 213, an external party file system B 214, a REST HTTP message N 215, an external HTTP request/response A 216, an external HTTP request/response B 217, a webhook to workflow A, 218 and/or a webhook to workflow B 219. The API gateway 111 is further coupleable to one or more connector(s), whereby the API gateway may be configured to request details from the connector(s) 220 for external communication and/or to receive external communication information from the connector(s) 220, for example responsive to the requested details. If one or more next steps exist for a workflow, the workflows module 240 may make a call to the API gateway 111 for the next step. A workflow run step completion status may be determined for each step until a workflow sequence is completed.

The API gateway 111 may be communicatively coupleable to the workflows module 240. The API gateway 111 may be configured to format and put data into one or more workflow queues for one or more workflow workers to process. The API gateway 111 may be further coupleable to an analytics engine 250 and may be configured to provide one or more sets of transaction information, for example to enable transaction logging by the analytics engine 250. The analytics engine 250 may be communicatively coupleable to the workflows module 240, with the workflows module 240 capable of providing one or more sets of workflow step information to the analytics engine 250, for example to provide workflow step logging by the analytics engine 250. The analytics engine 250 may be further configured to request one or more server-side data visualization requests from the memo charting and reporting engine 270 and to receive at least one of information and/or representation thereof responsive to the one or more requests. The workflows module 240 may be further coupleable to the EMPI engine 260. The workflows engine may be configured to request patient matching rules from the EMPI engine in an exemplary embodiment. The workflows module 240 may be configured to process one or more steps, to map fields, and/or to apply any filtering, translation, transformation, and/or patient matching rules depending at least in part upon profile settings.

FIG. 3 illustrates an exemplary embodiment of a partial functional block diagram according to aspects of the present disclosure. The system 300 includes one or more of a core UI module 340, and a core gateway 320, and a core API 150 according to aspects of the present disclosure. The core API 150 may be communicatively coupleable to at least one of the core UI and/or the core gateway 320. The core API 150 may be configured to communicate with the core UI via bidirectional publish/subscribe messaging over Transport Communication Protocol (TCP) in an exemplary embodiment, although other hardware- and/or software-based communication component or protocol may be used in various embodiments without departing from the spirit and scope of the present disclosure. The core API 150 may include a web socket server 322, an in-memory datastore client 324, and/or a web socket client 326. One or more of the web socket server 322, the in-memory datastore client 324, and the web socket client 326 may be used either alone or in part to communicate with one or more of the core UI 340, the core API 150, and/or a third-party system. The core API 150 may be configured to send and/or receive one or more messages 330 to/from a third-party, for example using bidirectional messaging such as bidirectional publish/subscribe over TCP. The core API 150 may be configured to communicate via one or more channels with a in-memory data server or store. The in-memory data server or store may be further communicatively coupleable to the core API 150, for example using one or more workers checking queues. In various embodiments, at least one of the connector microservice(s), workflows module, and/or authentication and authorization microservice of the core API 150 may be configured to convey information to the in-memory data server or store via the workers checking queues and according to one or more communication protocols or formats. The core API 150 may include one or more of a connector module 152, a workflow module 154, and/or an auth module 156 respectively coupleable to one or more workers checking queues.

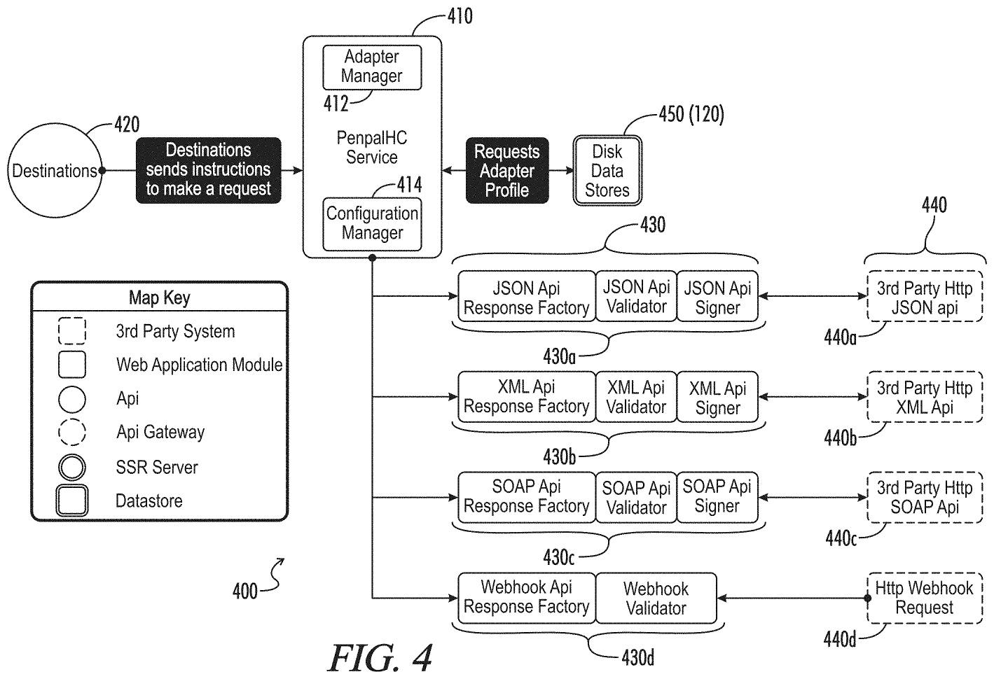

FIG. 4 illustrates an exemplary embodiment of a partial functional block diagram according to aspects of the present disclosure. The system 400 includes a PenpalHC service 410, a destinations module 420, one or more response interfaces 430, one or more APIs 440, and/or one or more disk data stores 450 (e.g., at least similar to encrypted disk data store 120). The PenpalHC Service 410 may include at least one of a connector manager and/or a configuration manager. The connector manager may be an HTTP connector manager as described herein with reference to FIG. 2, although other protocols may be implemented without departing from the spirit and scope of the present disclosure. Similarly, the configuration manager may be an HTTP configuration manager as described herein with reference to FIG. 2, although other protocols may be used without departing from the spirit and scope of the present disclosure. A connector microservice(s) module 420 (e.g., destinations module) may be communicatively coupleable to the PenpalHC service 410. The connector microservice(s) module 420 may be configured to transmit one or more instructions to the PenpalHC service 410 to form a request. The PenpalHC service 410 may be further communicatively coupleable to one or more disk data stores 450. In an exemplary embodiment, the PenpalHC service 410 is configured to request one or more connector profiles from at least one disk data store 450, and the at least one disk data store 450 is configured to provide at least one requested connector profile to the PenpalHC service 410 responsive to the request. In various embodiments, at least one disk data store 450 is a MariaDB or other SQL database, although other types, protocols, or formats of data storage may vary according to implementation.

The PenpalHC service 410 may be communicatively coupleable to one or more response interfaces 430, for example via one or more HTTPS connections via a wired connection, wireless connection, or combination thereof. The response interfaces 430 (which may also be referred to with respect to element number 180 herein) may include one or more of a JSON interface, an XML interface, a Simple Object Access Protocol (SOAP) interface, and/or a Webhook interface. In an exemplary embodiment, the response interface 430 may form at least a part of an API connector. The JSON interface 430a may include a JSON API response factory, a JSON API validator, and/or a JSON API signer. The XML interface 430b may include an XML API response factory, an XML API validator, and/or an XML API signer. The SOAP interface 430c may include a SOAP API response factory, a SOAP API validator, and/or a SOAP API signer. The Webhook interface 430d may include a Webhook API response factory and/or a Webhook validator. The JSON interface may be communicatively coupleable to a third-party JSON API 440a, for example an HTTP JSON API. The XML interface may be communicatively coupleable to a third-party XML API 440b, for example an HTTP XML API. The SOAP interface may be communicatively coupleable to a third-party SOAP API 440c, for example an HTTP SOAP API. The Webhook interface may be configured to output a Webhook request, for example an HTTP Webhook request 440d.

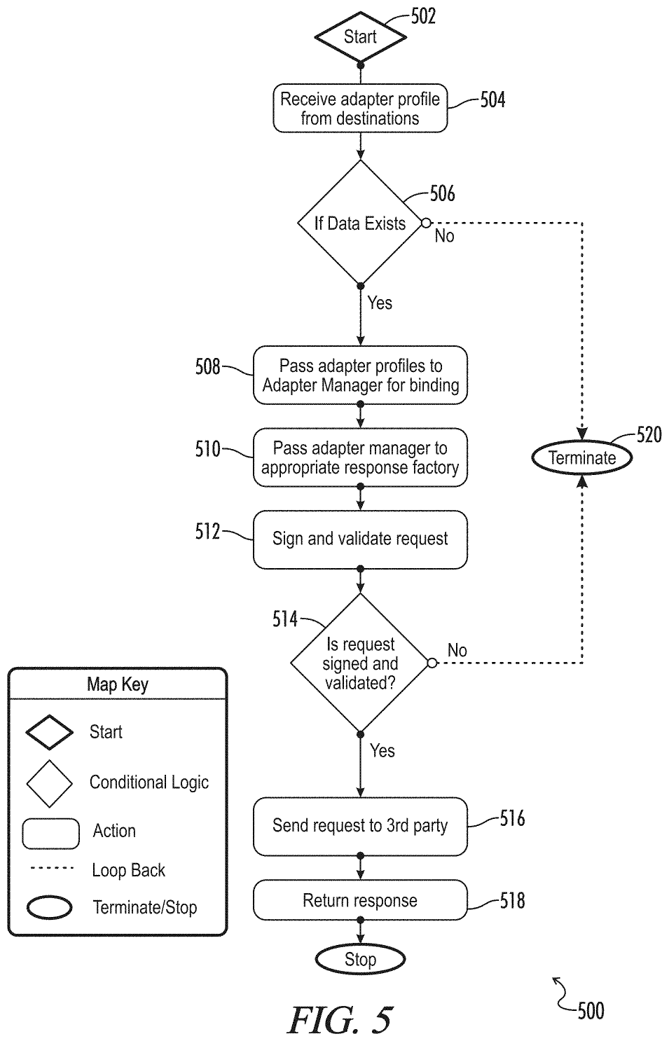

FIG. 5 illustrates an exemplary embodiment of a process for selectively binding a connector profile according to aspects of the present disclosure. The process 500 begins at a step 502. A connector profile is received from a destinations module at operation 504. It is determined at operation 506 whether data exists corresponding to the received connector profile. If data does exist, the process continues to operation 508 where the connector profile is passed to an adapter manager for binding. The process then continues to an operation 510 where a configuration manager corresponding to the connector profile is passed to an appropriate response factor. At operation 512 a validation request is signed and validated. It is then determined at operation 514 whether the request is signed and validated. If the request is not signed and validated, the process terminates at operation 520. Similarly, if it is determined at operation 506 that no data exists, the process terminates at operation 520. If it is determined at operation 514 that the request is signed and validated, the process continues to operation 516 where the request is sent to a third-party. The process then continues to operation 518 where a response is returned and the process 500 concludes.

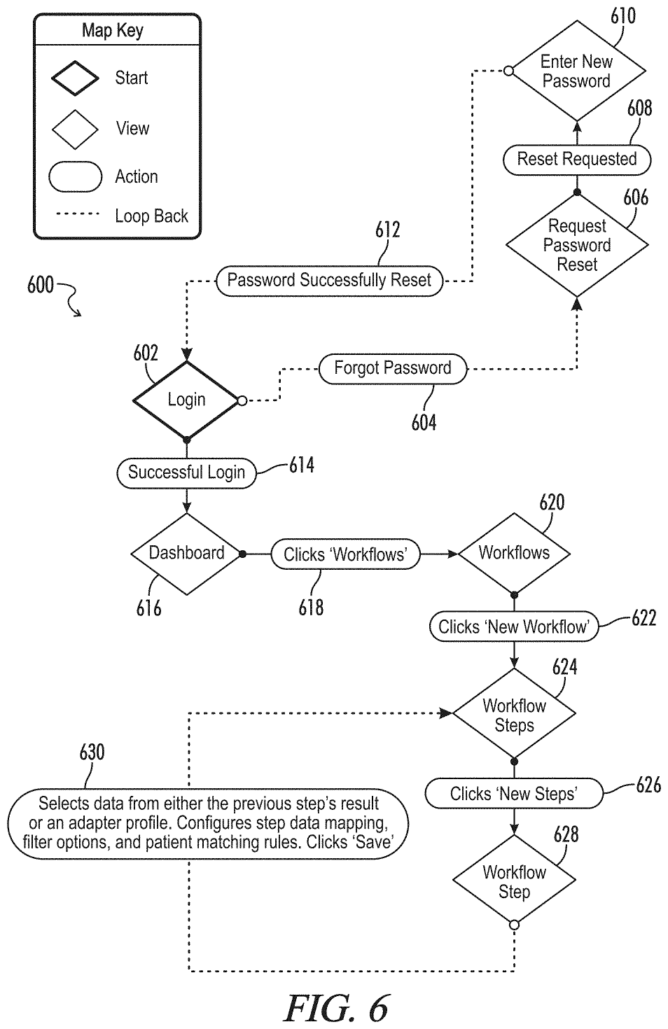

FIG. 6 illustrates an exemplary embodiment of user interaction with an interface and corresponding operations according to aspects of the present disclosure (e.g., as a workflow creation user flow diagram). The process 600 begins with a login interface being provided to a user at operation 602. The user may be provided with an option to rectify if they have forgotten their password at operation 604. If a user selects the forgot password option at operation 606, the user is presented with the ability to request a password reset at operation 608. The password reset may be accomplished, for example, by sending a link or identifier to the user, for example via electronic mail and/or SMS messaging which enables the user to provide a new password. Upon receiving the new password at operation 610, the system may store the new password associated with the user and may return to the login interface, optionally providing a password change successful message at operation 612. If a user's login authentication information matches stored or accessible user authentication data, the login is deemed successful at operation 614 and the user is provided with a dashboard interface at operation 616. Using the dashboard interface at operation 616, a user may create a new workflow at a workflow interface at operation 618 by selecting a “New Workflow” vi a workflows interface presented at operation 620 and/or similar element at operation 622. Each workflow step at operation 624 may be sequentially or non-sequentially generated by the user relative to one another in various embodiments. The user may, for example, select a “New Step” element or similar at operation 626 to create a new step within the workflow at operation 628. The user may then select data from either the previous step's result or a connector profile. Step data mapping may be configured, and filter operations and patient matching rules may be configured. The user may then store the step configuration, for example by selecting a “Save” element or similar at operation 630 and return to operation 624.

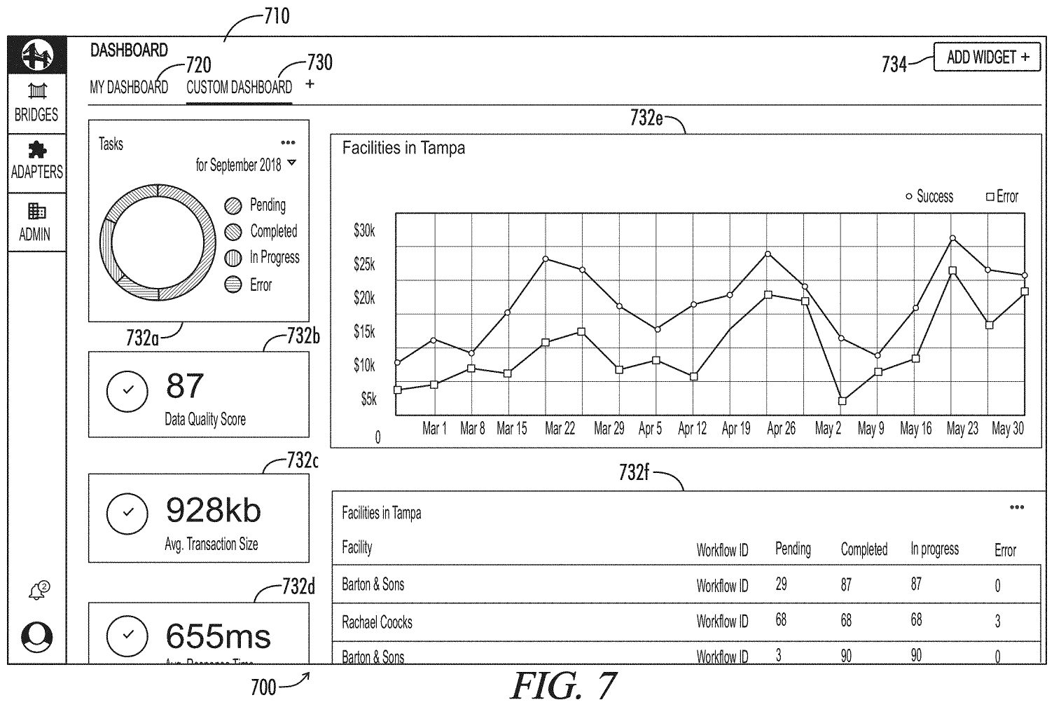

FIG. 7 illustrates an exemplary dashboard interface 700 according to aspects of the present disclosure. A dashboard 700 presented to a user may be a default dashboard selectable at element 720 including one or more predetermined presentation formats and/or information associated with one or more performance criteria for presentation to a user of the dashboard. Additionally or alternatively, a custom dashboard 730 may be optionally provided to a user including predetermined and/or user-customizable information for viewing. For example, in the custom dashboard of FIG. 7, a user may be presented with site or service-specific information such as facility information 732e, 732f for Tampa, Fla. or any other set of service and/or location information within the scope of the present disclosure. Dashboard information may include real-time and/or stored transaction data and may further include rating or scoring information 732 (e.g., 732a, 732b, 732c, 732d, 732e, 732f, etc.) associated therewith in various embodiments. A user of the dashboard may be provided to add, remove, or otherwise modify one or more components of the dashboard. An “Add Widget” button 734 may be provided to the user to selectively add one or more widgets or set(s) of interface information to the dashboard. Widgets selectable for the dashboard may be provided by the system described herein and/or may extend to third-party widgets or data.

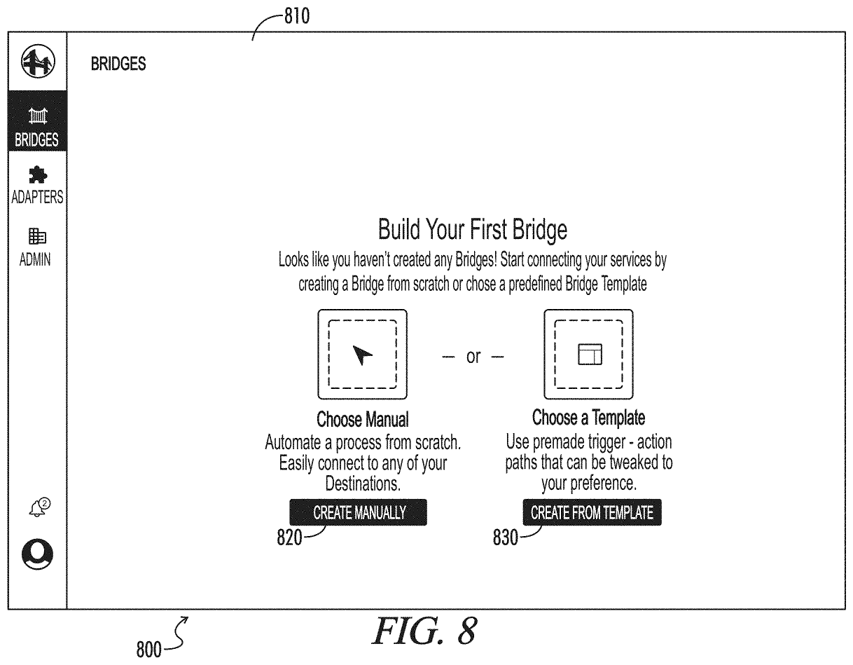

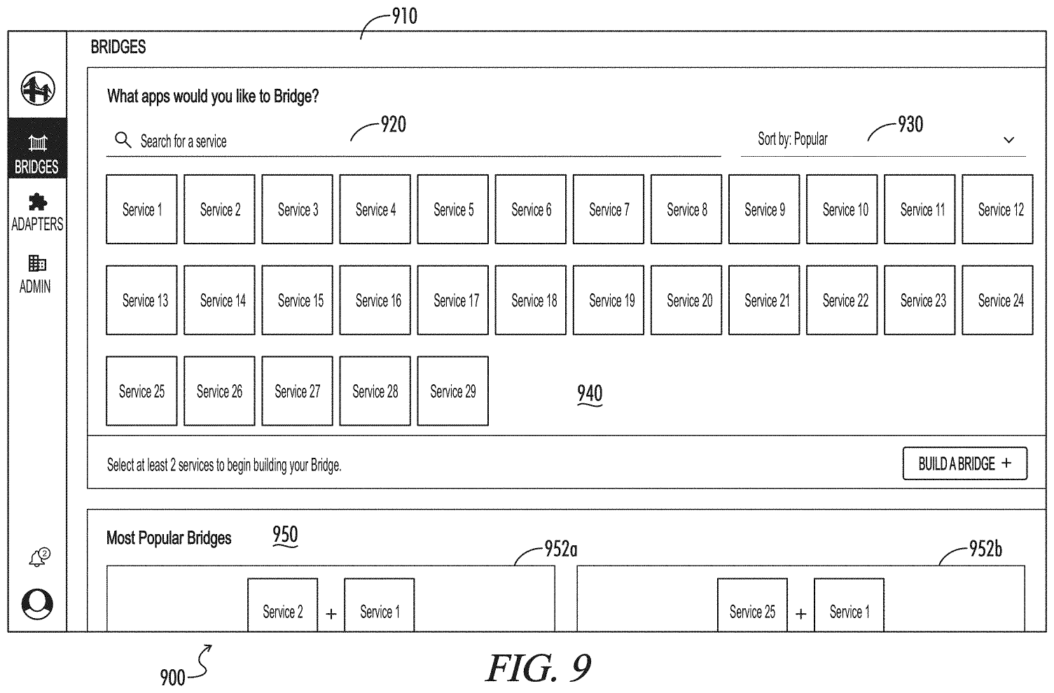

FIG. 8 illustrates an exemplary embodiment of a bridges interface 800 according to aspects of the present disclosure. In one exemplary embodiment, a user may select a Bridges icon or corresponding link available on the dashboard interface 810. As shown by FIG. 8, once selecting the Bridges icon, a user may be presented with options for creating a new manual 820 or template-based 830 bridge (for example upon a first selection of the Bridges icon). For situations where a user has previously created or selected at least one bridge, the user may optionally be presented with a list of previously established bridges and/or associated information. FIG. 9 illustrates an exemplary embodiment of a partial view of an interface 900 for a user to bridge two or more Vendors/apps according to aspects of the present disclosure. The user may select two or more Vendors/apps from a list of available Vendors/apps (which may be searching via search module 920 and/or sortable via sort selector 930, either of which may change a selection and/or arrangement of one or more of the list of available Vendors/apps corresponding to a selection in display area 940) and may optionally be provided with a list of most popular bridges 950 (e.g., via exemplary most popular bridges 952a, 952b), suggested bridges, new bridges, or any other set of predetermined or dynamically determined bridges.

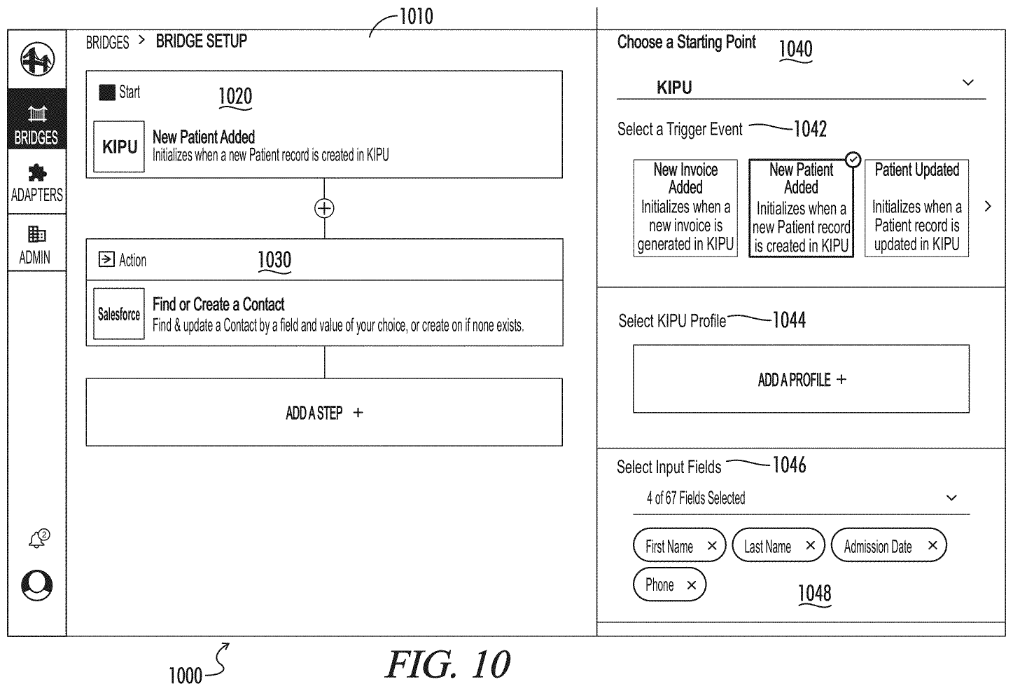

FIG. 10 illustrates an exemplary interface 1000 for manual bridge creation according to aspects of the present disclosure (e.g., as automated at least in part from a template in various embodiments). The bridge setup interface 1010 allows a user to create one or more steps in a workflow, each associated with one or more apps. For example, in the example illustrated by FIG. 10, a user selected a start point of KIPU as a starting app at section 1020, an action 1030, and the user selected the trigger event 1042 of a new patient added at starting point section 1040. Under this trigger event, the workflow initializes when a new patient record is created in KIPU. A user may be able to further specify a KIPU profile 1044 and may select one or more corresponding input fields 1046 associated with the workflow step (in this case, first name, last name, admission date, and phone number at section 1048).

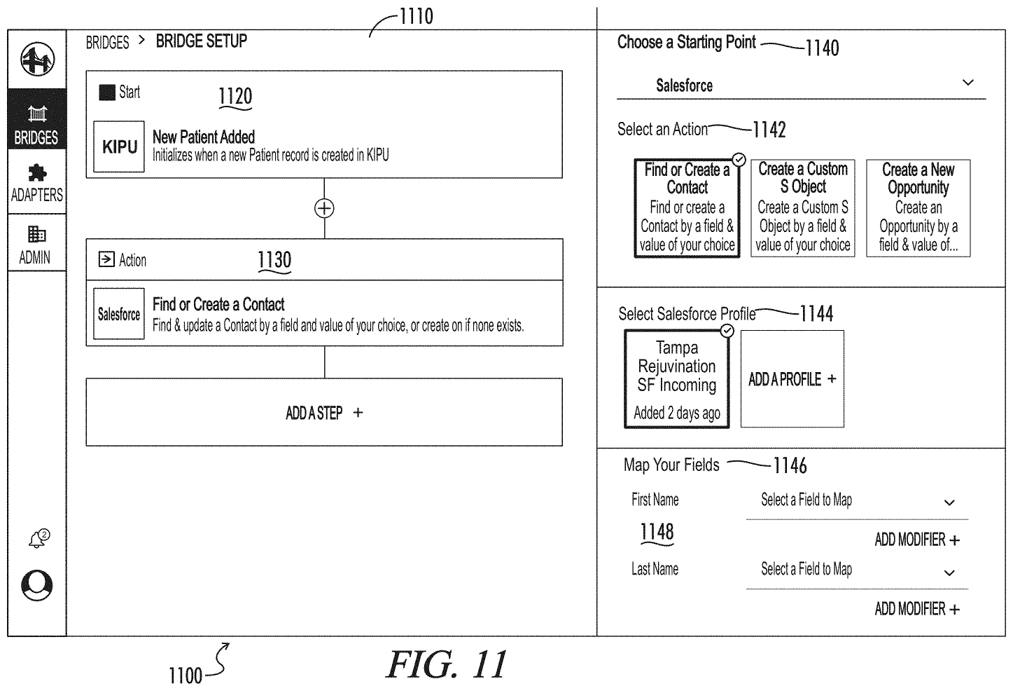

After setting the workflow trigger criteria, the user may select one or more action steps associated with the trigger. FIG. 11 illustrates an exemplary interface 1100 associated with an action step in the workflow of FIG. 10 (e.g., as optionally generated at least in part according to template details) according to aspects of the present disclosure. A user may be provided with the ability to select a destination/connector point 1140 corresponding to a trigger at section(s) 1120, 130 via a setup interface 1110, Salesforce in the example of FIGS. 10-11. The user may select an action 1144 to be performed responsive to receiving information from a previous workflow step and illustrated, for example, at section 1130. Here, the selected action is to find or create a contact based at least in part upon the input fields identified in FIG. 10. The action is designed to find or create a contact by a field and value of the user's choice. The user in this case selected a Salesforce profile associated with Tampa Rejuvenation. One or more fields may be mapped 1146 using the bridge setup interface, with the user being capable of adding one or more modifiers associated with one or more fields 1148.

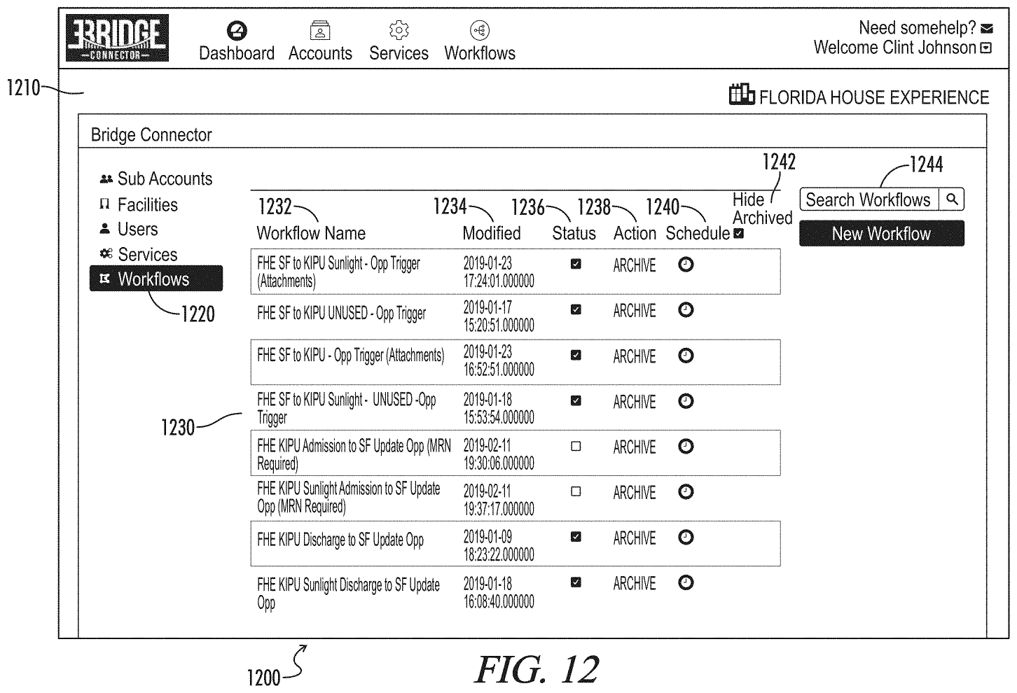

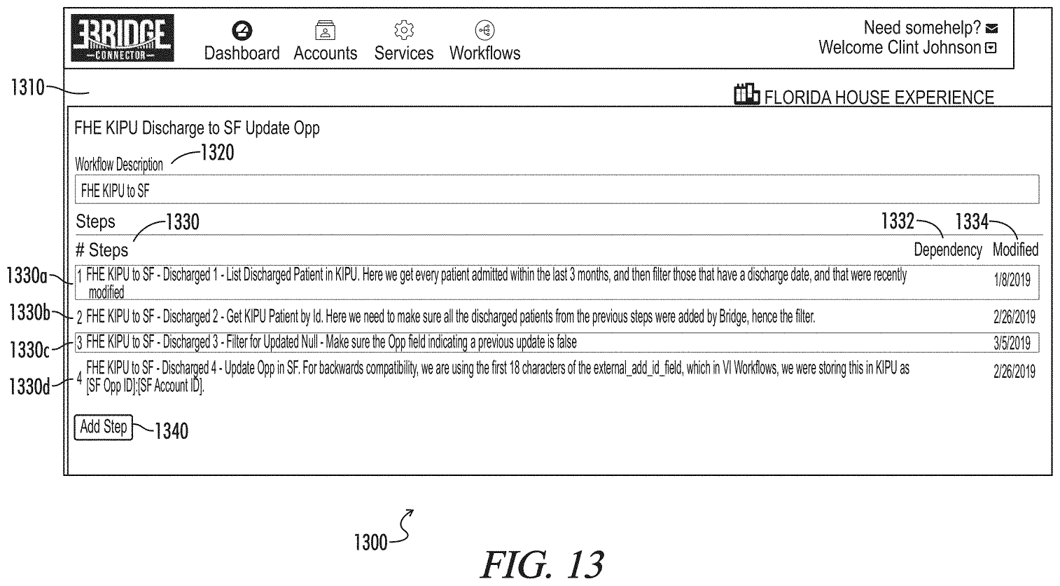

The workflow creation process using interfaces as illustrated by FIGS. 9-11 is further illustrated with regard to the exemplary interface provided by FIGS. 12-13. FIG. 12 illustrates an exemplary embodiment of an interface 1200 including a list of workflows 1230 associated with a user, a system, a location, an entity, a global listing, or any other criteria associated with one or more of the user and/or other entity. The list of workflows 1230 may include one or more columns, for example workflow name 1232, modification information 1234, status 1236, action 1238, schedule 1340, and/or hide archived 1342. A workflow search section 1244 may optionally be provided. A current indicator 1220 may be provided on by the interface 1200 to reflect selection of the workflow interface 1200.

If a user selects the FHE KIPU Discharge to SF Update Opp from the list of workflows 1230 illustrated in FIG. 12, the user may be provided with the interface 1300 of FIG. 13. The interface 1300 may include a workflow details interface 1310. A workflow description 1320 may be provided to identify the selected workflow. In the workflow illustrated by FIG. 13, a total of four operations 1330a-1330d are provided in the workflow 1330. It should be appreciated that one or more workflow operations may be performed in sequence linearly, out of order, and/or selectively performed based at least in part upon a previous workflow step and/or other criteria. One or more steps of the workflow may permit a user or workflow creator to provide a description of the operation of each step and/or the workflow as a whole. One or more of a dependency column 1332 and/or modification information 1334 may selectively be provided in relation to one or more workflow operations 1330a-1330d.

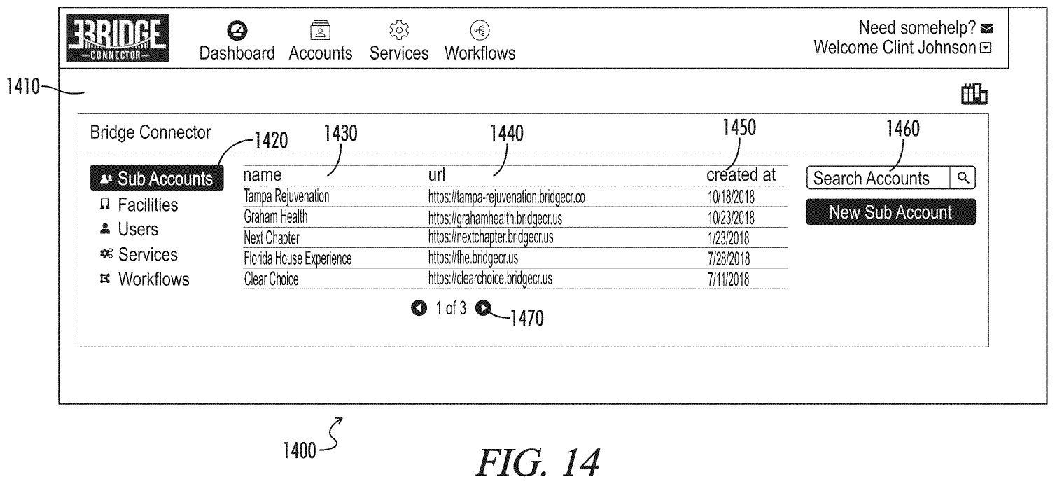

FIG. 14 illustrates an exemplary embodiment of a user interface 1400 identifying one or more sub accounts 1420 associated with a user (e.g., as a legacy mode account page) according to aspects of the present disclosure. In various embodiments, each sub account may be associated with a subset of systems, locations, or elements associated with the user, one or more of which may have separate and/or common workflows, services, users, and/or facilities. The sub-account interface 1410 may include one or more columns corresponding to at least one sub-account. For example, the sub-account 1410 may include one or more of a name column 1430, a URL column 1440, a creation information column 1450, and/or may include an account search section 1460. In cases where a plurality of sub-accounts do not fit on a single sub-account interface 1410, a page selector 1470 may be provided to move between pages of sub-accounts.

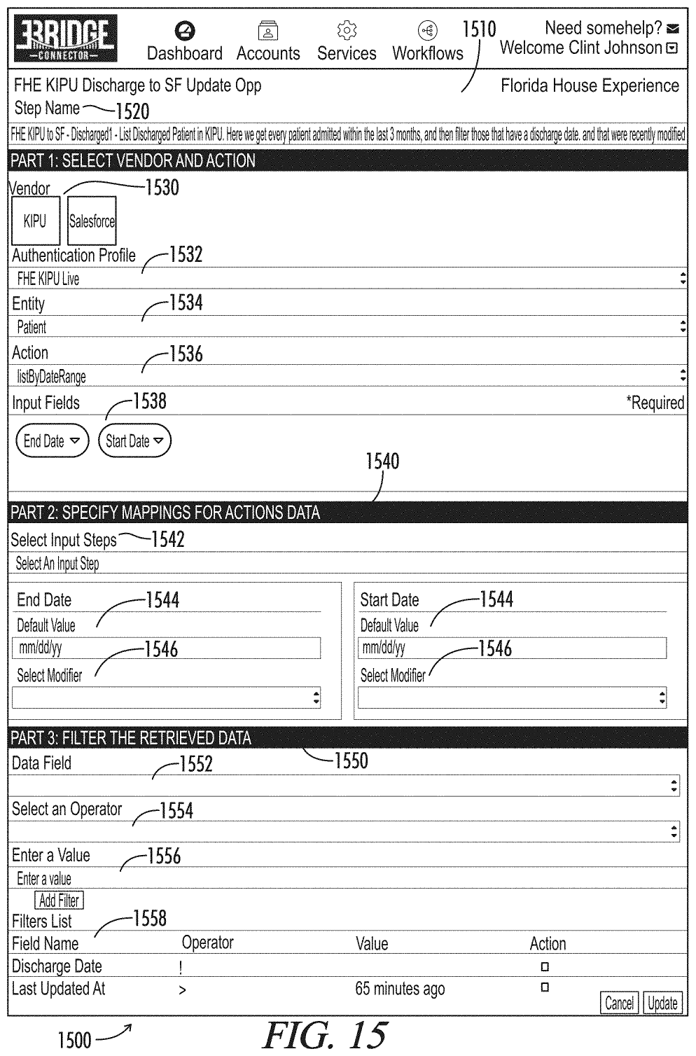

FIG. 15 illustrates an exemplary embodiment of an interface 1500 provided to a user according to aspects of the present disclosure, for example upon the user selecting a particular workflow step or requesting to create a new workflow step (e.g., as a legacy workflow step editing interface). A user may be provided with the ability to modify the operation name 1520 via the workflow operation setup interface 1510. Optionally in a section for permitting a user the select a Vendor/app 1530 and an action 1536, one or more vendors associated with one or more apps may be identified and/or selected by the user. An authentication profile 1532 may be selected for at least one vendor and/or app 1530. An entity type 1534 and action 1536 may selectable and/or identified via the interface. One or more input fields 1538 may be selected and/or identified. The user may specify mappings for actions data at section 1540. The user may select one or more input steps, start date(s) and end date(s) 1544, and/or modifiers 1546 associated therewith. The interface 1500 may further permit a user to create one or more filters for retrieved data via a filter section 1550. The user may specify a data field 1552, may select an operator 1554, and may enter a value 1556. A list of filters 1558 may be provided to the user via the interface 1500. The filters list may include information such as, but not limited to, a field name, an operator, a value, and/or an action.

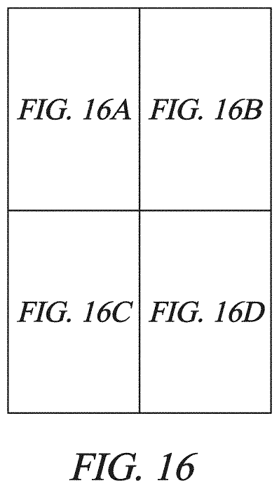

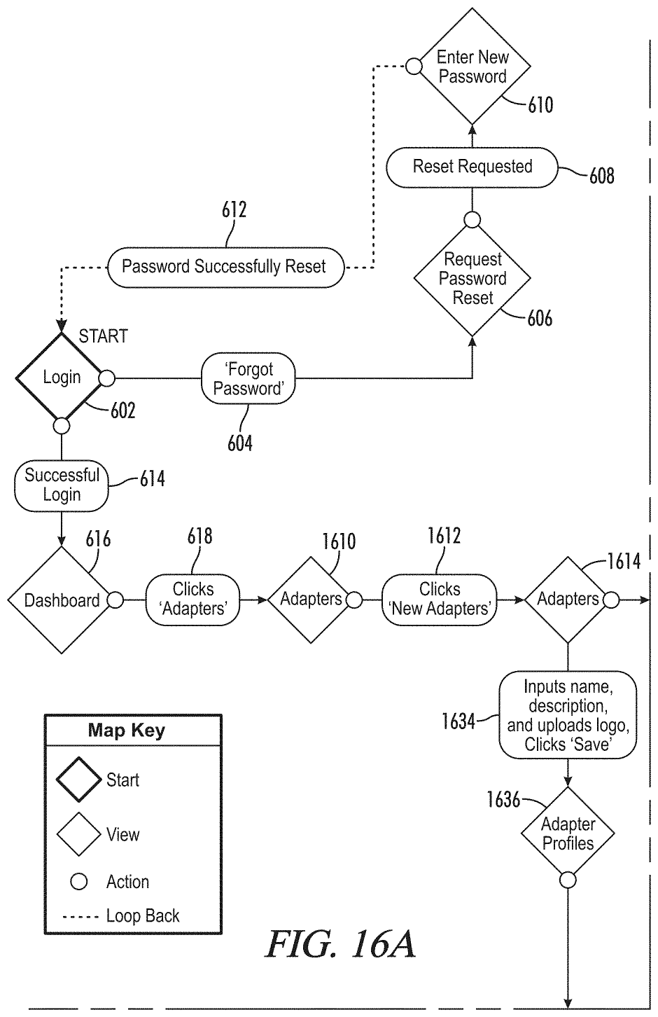

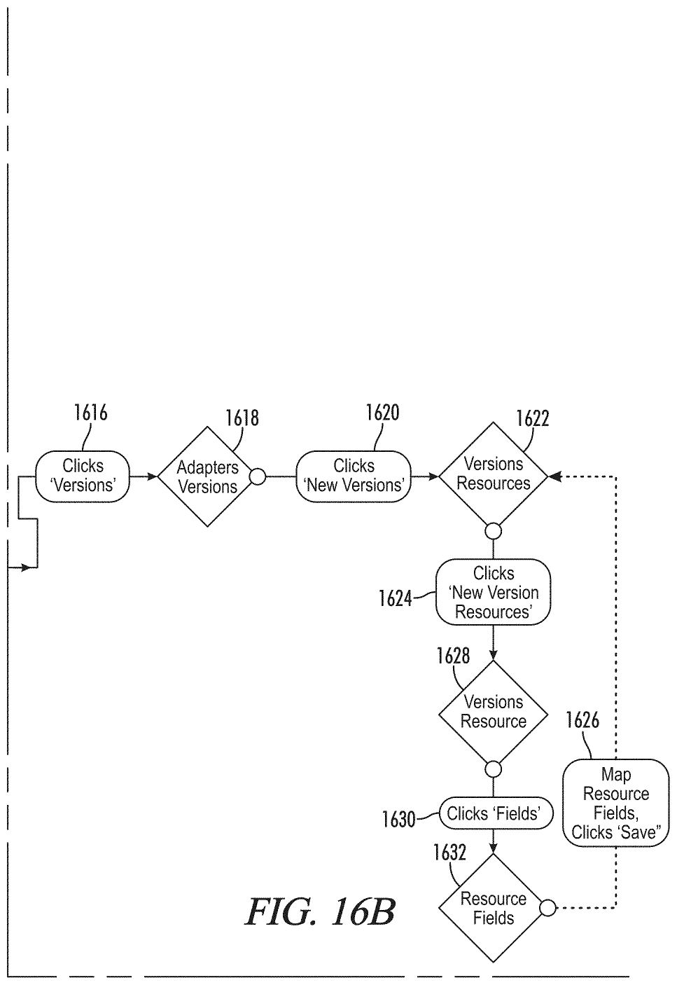

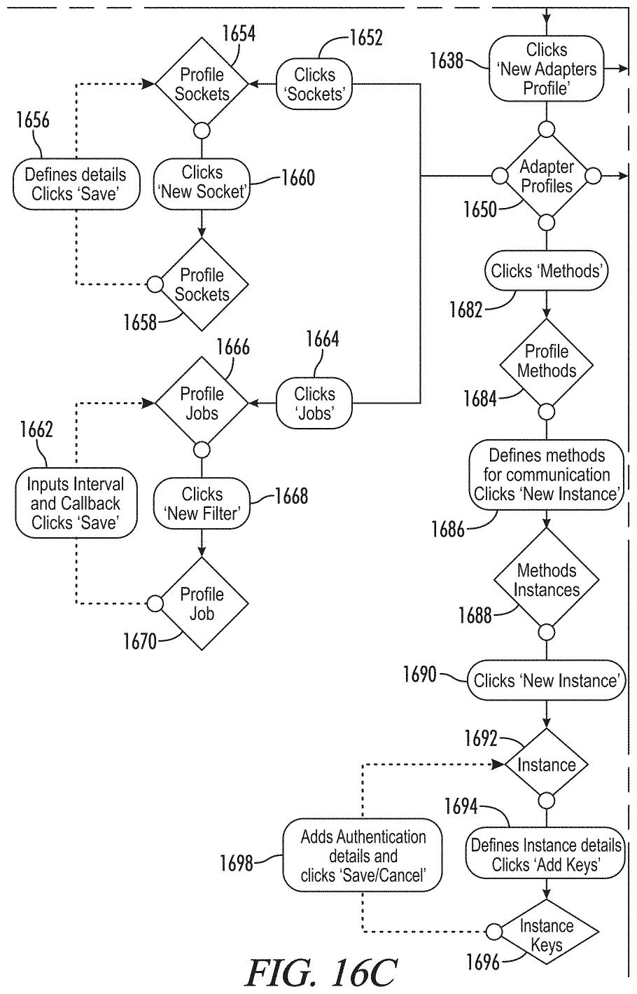

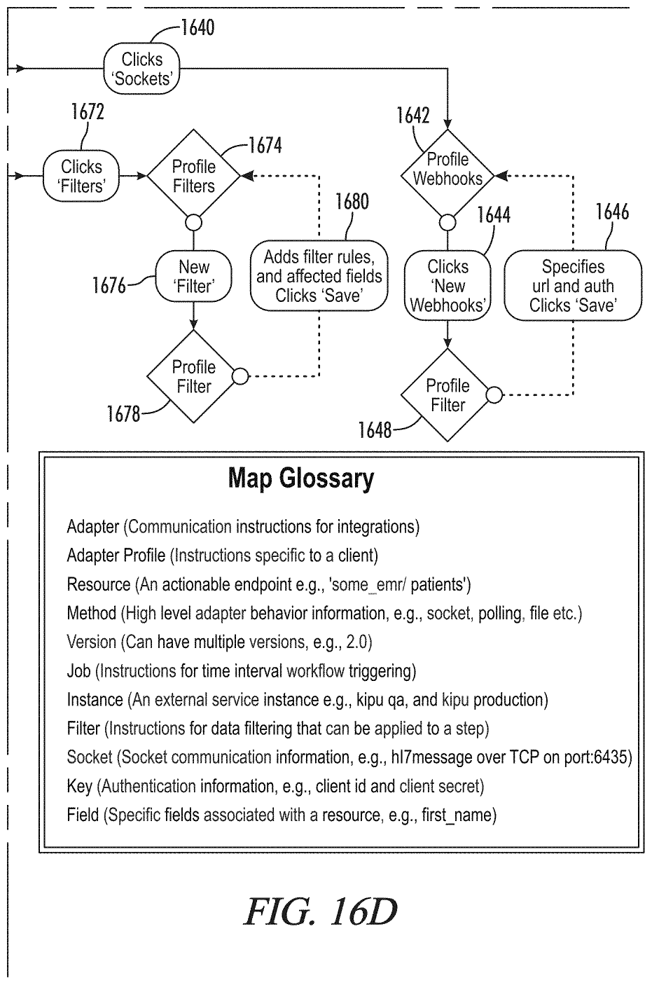

FIGS. 16 and 16A-D collectively illustrate an exemplary embodiment of a connector creation user flow according to aspects of the present disclosure (e.g., as a connector creation user flow diagram). The flow begins at a login operation similar to as described above with reference to FIG. 6. After landing at the dashboard, the user may select an “Connectors” element 1610 permitting the user to select to create a new connector 1612 at element 1610. The user may select a “Version” element 1616 to view connector versions 1618. The user may select a “New Version” element 1620 to view version resources 1622. The user may select a “New Version Resource” element 1624 or similar to view resource field 1628 and to map resource fields 1630, 1632 (e.g., by selecting a “Save” or similar element 1626). The user may input one or more of a name or description in the new or existing connector configuration interface at operation 1634. The user may selectively create a new connector profile 1638 at the connector profiles 1636. The connector profile may have at least one associated socket profile which may be selected by a user at operation 1640, 1652 from the connector profile 1650. The user may be permitted to create a new set of socket information for a connector profile and to save that socket information with the connector profile by selecting a create new socket option 1660 via the profile sockets 1654 and to save the same at operation 1656. The user may be permitted to create one or more new jobs associated with the connector profile by selecting a new job selector 1668 via the profile jobs 1666, such as input interval and callback information 1662. The user may enter one or more sets of new profile job information and to save such along with the connector profile via profile job 1670. The user may select a filter option 1672 to associate one or more filters with a connector profile 1674, for example by selecting a “New Filter” element 1676 or similar, and by providing one or more filter rules and/or affected fields, and by saving such as a profile filter 1678 with the connector profile via operation 1680. The user may further create one or more new Webhooks 1642, for example by selecting a new webhook selector 1676 and providing one or more sets of URL and/or authentication information 1646. The Webhook information may then be stored with the connector profile as a profile webhook 1678.

A user may interact with a profile methods interface 1684 to define a method for communication, for example by selecting a new instance selector 1686. A method instances operation 1688 may then be used to permit a user select a new instance selector 1690. Using an instance interface 1692 a user may define instance details and selectively activate an add keys operation 1694 before providing an instance keys interface 1696. Authentication details may be added via the instance keys interface 1696 and may be added upon selection of a save/cancel selector 1698 by the user.

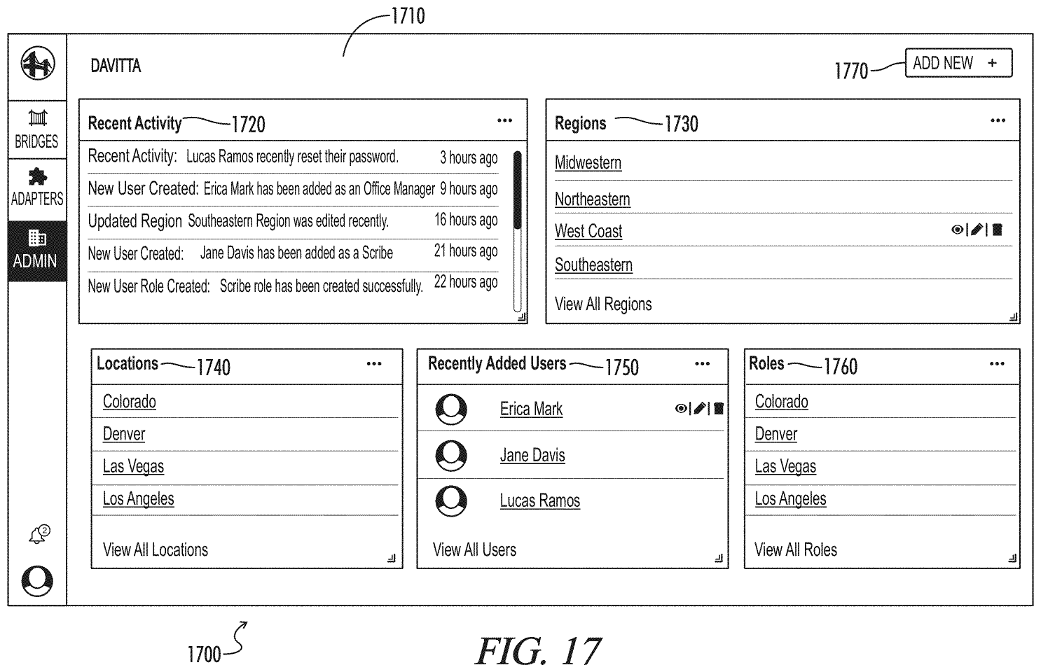

FIG. 17 provides an exemplary embodiment of an administration interface 1700 provided by aspects of the present disclosure. The administration interface 1700 includes a portal 1710 having one or more of a recent activity section 1720 a regions section 1730, a locations section 1740, a user section 1750, and/or a roles section 1760. One or more of the sections provided by the portal 1710 may permit administration of an organization and/or users according to one or more modifiable criteria.

FIG. 18 illustrates an exemplary implementation of workflow processing according to aspects of the present disclosure. The process 1800 begins with an API gateway 111 transmitting a connector profile and/or authentication or authorization to a workflows module at operation 1802. The workflows module 240 selectively transmits a notification to the API gateway 111 if a next step exists in the sequence at operation 1804. The notification provided by the workflows module 240 to the API gateway 111 may be generated according to workflow results, the results being used by the workflows module to generate and transmit a call to the API gateway 111 for the next step. The workflows module 240 may be configured to process a one or more workflow steps of a sequence at operation 1806. Step data may be mapped based at least in part upon profile mapping (e.g., connector profile mapping) at operation 1808. Step data may then be filtered based at least in part upon connector profile filter rules at operation 1810. The process continues by translating step data based at least in part upon one or more connector profile translation rules at operation 1820. Step data may then be transformed based at least in part upon one or more connector profile transformation rules at operation 1830. Patient matching may then be attempted based at least in part upon one or more EMPI rules at operation 1840. It is then determined by a third-party system whether a patient match confidence is greater than or equal to a predetermined or dynamically determined setting associated with a connector profile (e.g., included as a setting in a connector profile) at operation 1860. If it is determined that the patient match confidence is less than the setting, the workflow may continue to an “instruction needed” queue at operation 1880 to obtain one or more interactions to complete the workflow step. The process then continues to workflow step run complete operation 1870, whereby one or more results of a workflow step are selectively returned to the workflows module and optionally provided to a next step until a particular workflow sequence is completed. The process may return previous step results to a next step until the workflow sequence is complete at operation 1890.

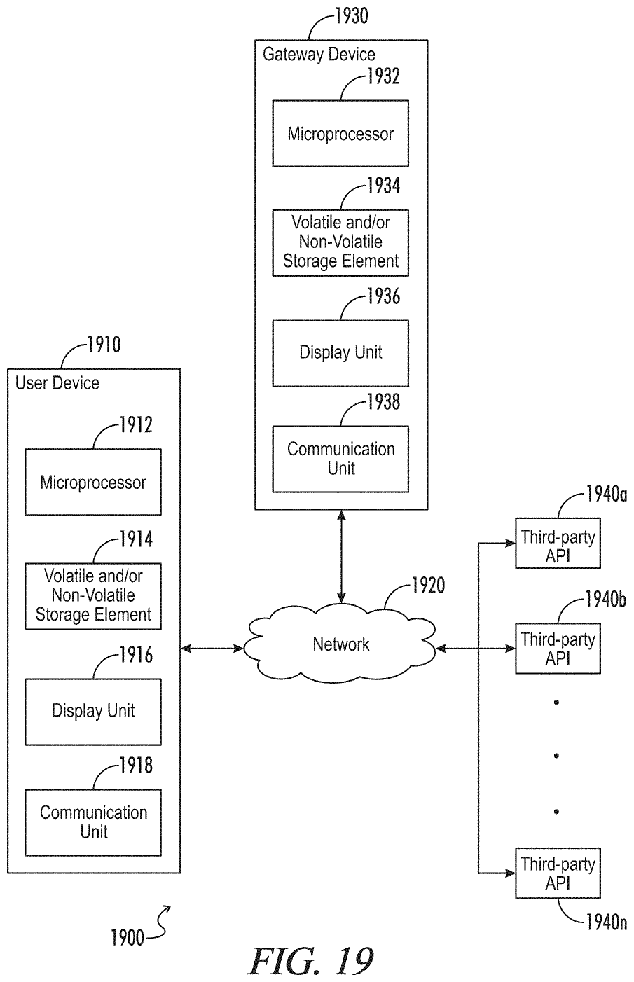

FIG. 19 illustrates an exemplary embodiment of a partial block network diagram according to aspects of the present disclosure. The system 1900 is a simplified partial network block diagram reflecting a communicative configuration implementable according to the present disclosure. The system 1900 includes a user device 1910 coupleable to a network 1920, a gateway device 1930 coupleable to the network 1920, and one or more third-party APIs 1940 coupleable to the network 1920. The user device 1910 may implement, in whole or in part, at least a portion of the functionality of the core client previously described herein. The gateway device 1930 may be configured to correspond to at least one of the core gateway 320, the core API 150, and/or the core UI 340, either as a standalone device or in combination with at least one other external component either local or remotely communicatively coupleable with the gateway device 1930.

In one exemplary embodiment, the network 1920 includes the Internet, a public network, a private network, or any other communications medium capable of conveying electronic communications. Connection between elements or components of FIG. 19 is configured to be performed by wired interface, wireless interface, or a combination thereof, without departing from the spirit and the scope of the present disclosure. At least one of the user device 1910 and gateway device 1930 may include a communication unit 1918, 1938 configured to permit communications via the network 1920.

In one exemplary operation, at least one of user device 1910 and/or gateway device 1930 is configured to store one or more sets of instructions in a volatile and/or non-volatile storage element 1914, 1934. The one or more sets of instructions may be configured to be executed by a microprocessor 1912, 1932 to perform operations corresponding to the one or more sets of instructions.

In various exemplary embodiments, at least one of the user device 1910 and/or gateway device 1930 is implemented as at least one of a desktop computer, a server computer, a laptop computer, a smart phone, or any other electronic device capable of executing instructions. The microprocessor 1912, 1932 may be a generic hardware processor, a special-purpose hardware processor, or a combination thereof. In embodiments having a generic hardware processor (e.g., as a central processing unit (CPU) available from manufacturers such as Intel and AMD), the generic hardware processor is configured to be converted to a special-purpose processor by means of being programmed to execute and/or by executing a particular algorithm in the manner discussed herein for providing a specific operation or result.

One or more computing component and/or functional element may be configured to operate remotely and may be further configured to obtain or otherwise operate upon one or more instructions stored physically remote from the computing component and/or functional element (e.g., via client-server communications and/or cloud-based computing).

At least one of the user device 1910 and gateway device 1930 may include a display unit 1916, 1936. The display unit 1916, 1936 may be embodied within the computing component or functional element in one embodiment and may be configured to be either wired to or wirelessly interfaced with at least one other computing component or functional element. The display unit may be configured to operate, at least in part, based upon one or more operations of the described herein, as executed by the microprocessor 1912, 1932.

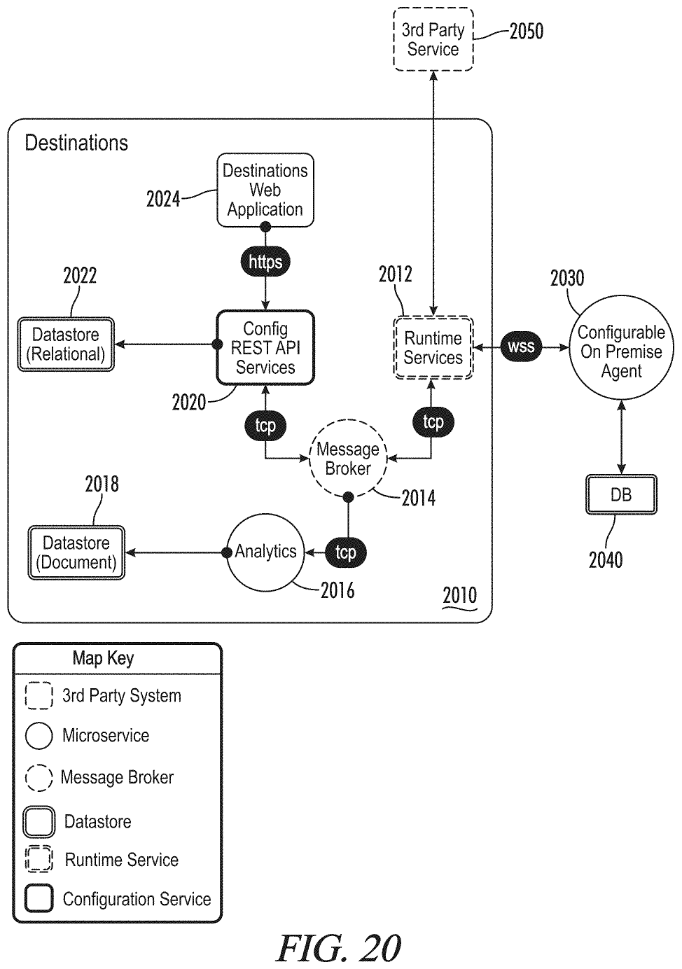

FIG. 20 illustrates an exemplary embodiment of a partial block diagram of a system according to aspects of the present disclosure. The system 2000 includes a destinations entity 2010 coupleable to a network (e.g., network 1920), a configurable on-premise agent 2030 coupleable to the network, a database 2040 communicatively coupleable to the configurable on-premise agent 2030, and/or a third-party service 2050 coupleable to the network 1920. The destinations entity 2010 includes a runtime services module 2012, a message broker 2014, an analytics engine 2016, a datastore 2018, a configuration REST API services module 2020, a datastore 2022, and a destinations web application 2024. The on-premise agent 2030 and at least one third-party service 2050 may be configured to communicate with the runtime services module 2012 of the destinations entity 2010. The on-premise agent 2030 may be coupled to a database 2040, either locally or remotely, or according to a combination thereof. The on-premise agent 2030 may be configured to communicate with the runtime services module 2012 using a WebSocket (WSS) protocol (e.g., via the network 1920).

The message broker 2014 may be perform at least one operation previously described herein with reference to message broker 140. The message broker 2014 may be communicatively coupleable to the runtime services module 2012, the analytics engine 2016, and/or the configuration REST API services module 2020 via at least one Transmission Communication Protocol (TCP) connection. Analytics module 2016 may be configured to perform one or more operations previously described herein with reference to the Analytics Services 115. The datastore 2018 may be configured to communicate with the analytics module 2016 and may be configured to store at least a portion of one or more documents or set(s) of information associated therewith, either temporarily or permanently. The configuration REST API services 2020 may be communicatively coupleable to the datastore 2022. The datastore 2022 may optionally be configured as a relational database. The datastore 2022 may be configured to store at least a portion of one or more documents or set(s) of information associated with the configuration REST API services, either temporarily or permanently. The destinations web application 2024 may be configured to communicate with the configuration REST API services via a secure HTTP protocol. Although illustrated as each being contained within the destinations entity 2010, it should be appreciated that one or more of the runtime services 2012, the message broker 2014, the analytics module 2016, the datastore 2018, the configuration REST API services 2020, the datastore 2022, and/or the destinations web application 2024 may be located locally or remotely to the destinations entity 2010, either in whole or in part, and in a physical sense as well as a virtual sense.

The features of FIG. 20 may offer certain advantages when compared to the system illustrated by FIG. 1. For example, an issue where configuration tasks and user interaction on the system might become bogged down when the workflow runtime engine operates at higher loads may be prevented or reduced. If not reduced or eliminated, this issue could lead to more significant issues such as a crashing runtime (e.g., as an infinite loop situation on the configuration side).

Unlike previous systems, implementations consistent with FIG. 20 may permit benefits in terms of scaling, as most of the work being done is not configuration, but rather runtime. In accordance with aspects of the present disclosure, users may log in and create a workflow which may then be configured to run without user interaction for long periods of time (e.g., years at a time). By implementing a system as described with reference to FIG. 20, the system gains the ability to horizontally scale configuration services separately from runtime. Performance benefits may thus be gained from scaling runtime services without wasted cost scaling services that did not get as much load. In a similar vein, at least a portion of analytics code may be implemented by the analytics module 2016 as optionally separated out into its own stack, and these all may communicate to each other via the message broker 2014 in real-time. Furthermore, the datastore 2018 may be separated from the datastore 2022. In various embodiments, the datastore 2018 may be an Online Transactional Processing (OLTP) database which is physically and/or virtually distinguished from the datastore 2022, which may be operating as an Online Analytical Processing (OLAP) database. By bifurcating the datastore 2018 and datastore 2022 one or more analytics queries are simplified and thereby running one or more dashboards are improved by leveraging the OLAP database and cutting down on costly join queries.

As compared to the system as illustrated, for example, by FIGS. 1-4, the embodiment illustrated by FIG. 20 is simplified and includes runtime services grouped together into a single representative “Engine.” One or more runtime services consistent with the present disclosure may include a runtime set up to run workflows in a linear fashion, such as f(f(g( )) to process a step, as illustrated and described herein, for example with reference to PenPalHC. Such linear workflow runtime process provides natural orchestration of functions. However, implementations consistent with the present disclosure may additionally or alternatively utilize process chains/the actor model, where each workflow is a supervisor process spawned by runtime, then spawns individual child process for each step as it processes the workflow iteration. When steps complete their process may die, similarly when all of them complete the workflow process completes and may die. Processes in this regard are very light and the system can easily scale to hundreds of millions, thereby providing greater scalability and fault tolerance. Implementations consistent with the present disclosure may further include systems leveraging distributed computing, which permits workflows to be scaled across multiple nodes, allowing further scalability and fault tolerance.

In an exemplary embodiment when a workflow is triggered to run the workflow spawns its own process, sometimes referred to as a supervisor process. This supervisor process may be responsible for spawning and monitoring step processes. As the workflow process starts the orchestration it spawns a new process to run each step, and that step then performs its action, returns the results and dies (e.g., as described herein with reference to a PenpalHC flow). This workflow process continues until all the steps have finished and it can gracefully die.

Many benefits come from enhancements to the workflow level runtime described, for example, in relation to FIG. 20, the first being process isolation. Workflows and steps cannot negatively impact others. In the worst case where one is unresponsive, thresholds will be hit and retry strategies of the workflow will execute without any other process knowing there was a problem with that specific instance.

Another benefit of implementations consistent with FIG. 20 is distribution. If the message broker is determined to be filling with unprocessed work, another instance of the engine may be quickly spooled, which may start pulling work from the message broker in parallel. the same strategy may be applied to three or any number of engine instances added, barring that broker limitations are not encountered before they become useful.

A further advantage of implementations consistent with the embodiment illustrated by FIG. 20 relates to fault tolerance. If an engine node drops for some unforeseen reason, it may be configured to rely on existing nodes to process the load from the dropped node and to assess one or more metrics to decide if another node should be spooled up to bring the system back to 100%.

Mapping/AI

As described herein, mapping may include providing an AI component configured to provide one or more suggested source-to-destination predictions for one or more connectors on the platform. In one exemplary embodiment, the AI component is configured to provide predictions for all connectors on the platform. To assist in providing predictions, the system may rely on the fact that all connectors must attempt to map to the common data model (e.g., based on the latest healthcare data specification) plus a possible modification via custom fields for things outside the model. Because each connector may be mapped to the data model, there is an inherent gain in source-to-destination mapping and mutation prediction for any connector represented as: Source->Data Model->Destination.

Simple Example: Patient First Name

Assuming that the source connector's first name field is keyed as “first” and that the destination connector's first name field is keyed as “first_name”, with the data model calls it “f_name.” Thus for source and destination we have: {“first”: “John” . . . } and {“first_name”: “John” . . . }. Since the source connector object has a mapping template of {“first”: “f_name” } and the destination connector object has a mapping template of {“first_name”: “f_name” }, we can then say {“first”: “f_name”: “first_name”}->{“first”: “first_name” }->{source: destination}.

This mapping approach allows systems consistent with the present disclosure to guess or deduce the mapping of any possibility the connector library might have. The mutation part systems consistent with the present disclosure may work in a similar manner where each and every connector has a transformation object when created. This object may provide the transformation required for that field to match the data models as well as the reverse treatment.

Simple Example: Patient Weight

Assuming the source connector's system uses weight in kg for patients and that the destination's system uses weight in lb for patients, with the bridge data model using lb. Each connector may have translation mappings that would look like this: source={“kg”: “lb”} and destination={“lb”: “lb”}. This is enough information for the system to convert kg to lb when extracting data from the source check what the destination connector uses and prepare for load.

This also works for other operations such as string manipulation with custom input for cases like gender {“m”: “male”} {“Male”: “male”}, date/time, data encoding, and more as we keep adding them.

A sidecar may be used for sets of data that are not represented by a common data model and may be surfaced to a user thereby providing a better user experience.

Implementations consistent with the present disclosure may be effective to reduce the amount of interaction and data needed from the user. Users may be permitted to configure on-premise database extractions from the Destinations entity 2010, allowing communication with systems that are not otherwise capable.

A destinations user may choose to configure an on-premise connector and may be prompted for basic database connectivity information of the system they are trying to access as part of the process. The user may supply the connector with actions which may be queries they would like to run against that database. The user may download an agent and install the agent on a server with access to the desired database. As the user builds workflows, he/she can select the on-premise connector and one or more database actions to run in a designated step. Now this data from the on-premise server is usable in workflows.

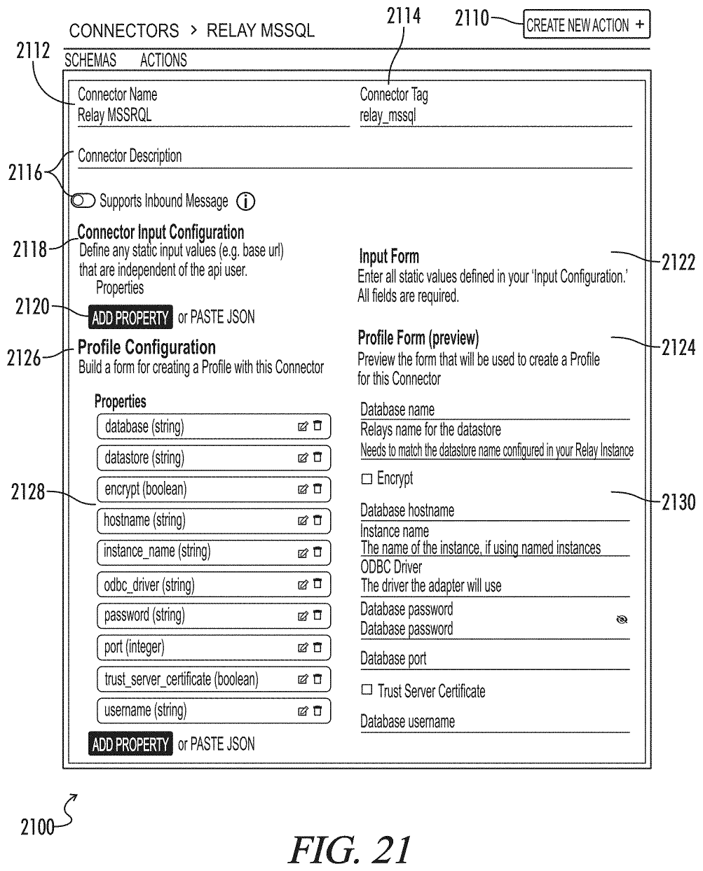

FIG. 21 illustrates an exemplary embodiment of an interface 2100 for creating or editing a connector according to aspects of the present disclosure. The interface 2100 includes a create new action element 2110. A connector name 2112 and connector tag 2114 may be provided by a user and/or may be automatically generated or combination thereof. A connector description and inbound message option 2116 may be provided in association with a particular connector. A connector input configuration section 2118 may include one or more settings or options 2128 associated with a configuration for a particular connector. Properties may include, but are not limited to, a database, a datasource, an encryption setting, a hostname, and instance name, an ODBC driver, a password, a communication port, a trust server certification setting or information, a username, or any other setting or information useable by or in conjunction with systems disclosed herein. An input form 2122 may be used to specify or identify one or more static values used to create a profile for a particular connector. A profile form 2124 may be used to create a profile form for a particular connector. One or more settings or values 2130 may be used, for example corresponding to one or more of the settings or options 2128.

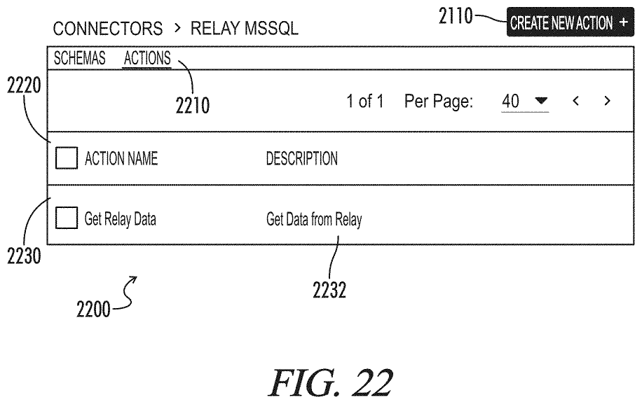

FIG. 22 illustrates an exemplary embodiment of an interface 2200 for viewing or specifying one or more actions for a connector according to aspects of the present disclosure. The interface includes the create new action element 2110, an actions selector 2210, an action information section 2220, a get relay data selector 2230, and a data description section 2232.

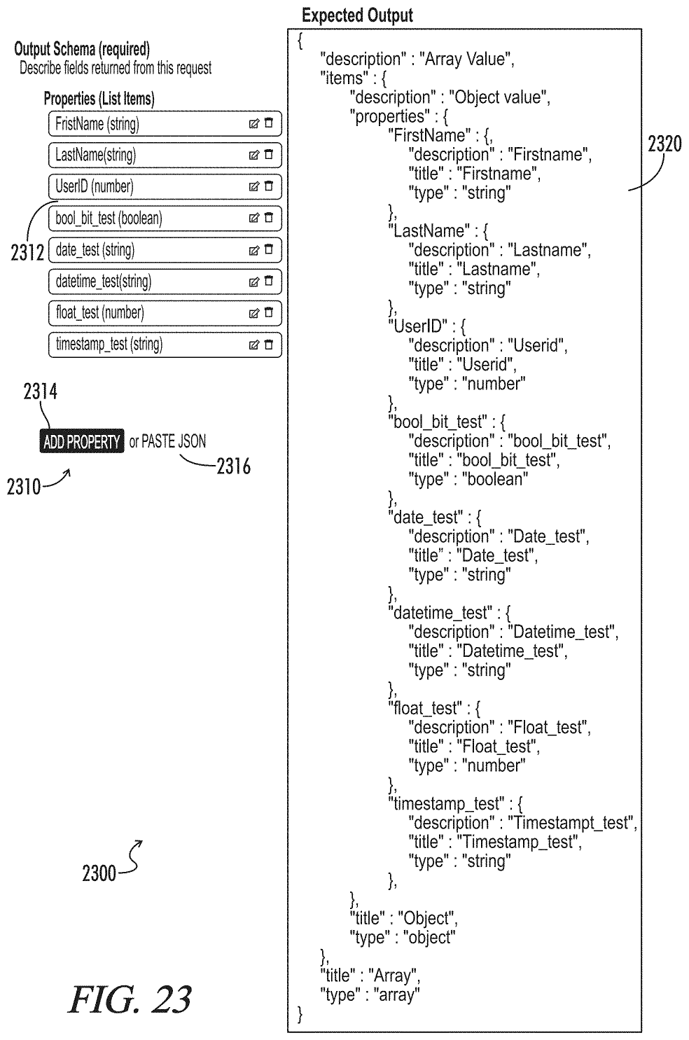

FIG. 23 illustrates an exemplary embodiment of an output schema interface 2300 according to aspects of the present disclosure. The interface 2300 includes a properties list 2310 and an optional expected output section 2320. The properties list includes a list of one or more properties 2312, an add property element 2314, and a paste JSON element 2316. The expected output section 2320 may visually indicate at least a portion of code corresponding to at least one property 2312 of the list of properties 2310. This may assist a user in connector creation, modification, or maintenance without having to write at least a portion of the code in the expected output section 2320 and/o may provide programming guidance to a user in relation to a particular connector for creation, modification, or updating, or for use with a new or similar connector to a previously created connector. One or more portions of the interface 2300 may be generated or accessed after selecting the Get Data from Relay option of FIG. 22 and/or may surface to the workflow building after the connector is verified (e.g., via the interface illustrated by FIG. 23).

One or more new connectors (e.g., as third-party system representations) may be created and/or communicated with via the Destinations element 2110 without the need to write custom code based on selection of one or more basics of the desired connector by the user.

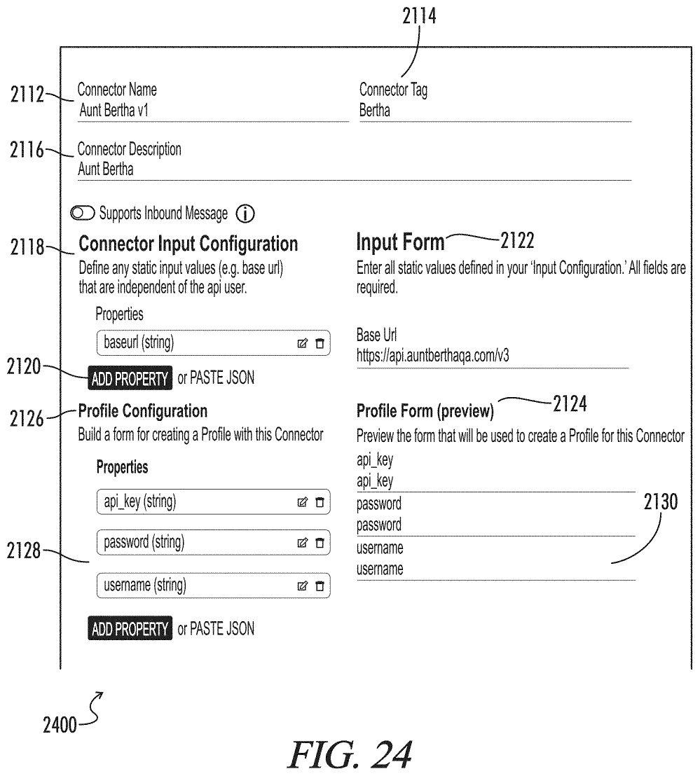

FIG. 24 illustrates an exemplary embodiment of a complete version of an interface of FIG. 21 according to aspects of the present disclosure. The interface 2400 includes a completed version of the interface 2100 provided by FIG. 21. The completed interface 2400 permits the system to allow a user to define both the general connector details that are shared by all workflows using the connector as well as profile-level details which are specific to their account on the Destinations platform. That is to say that users may define one or more actions describing how the system will communicate.

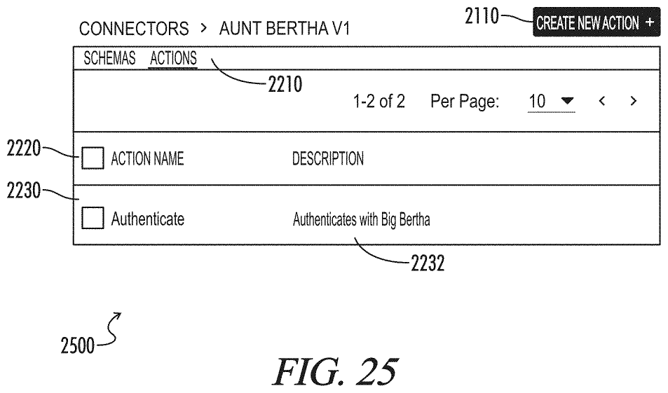

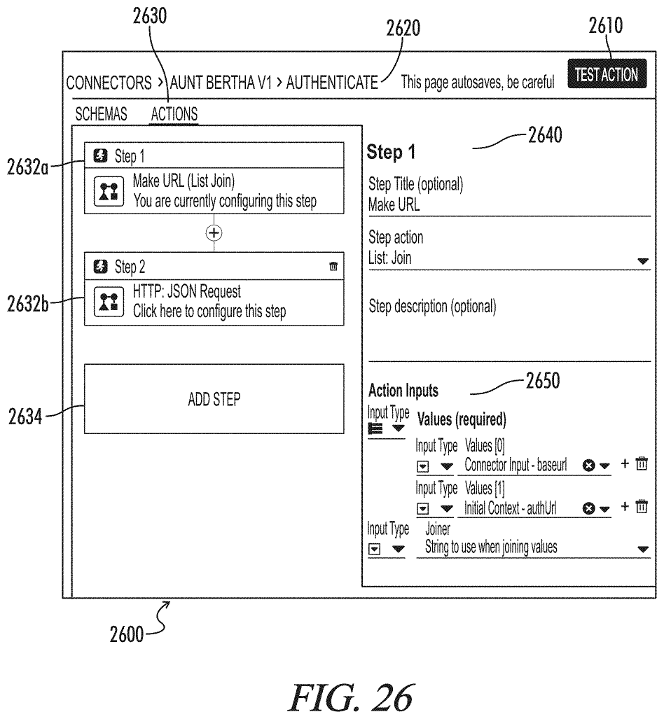

FIG. 25 illustrates an exemplary embodiment of an interface of FIG. 22 according to aspects of the present disclosure. A user may define at least one action, provide workflow information, and expected detailed required for use in a workflow as illustrated by FIG. 26.

FIG. 26 illustrates an exemplary embodiment of an interface 2600 for creating an authentication flow for a connector according to aspects of the present disclosure. The interface 2600 includes a test action element 2610. In the embodiment illustrated by FIG. 26, a user is provided with the ability to create, edit, or modify an authentication process 2620 access flow 2630. The flow illustrated by FIG. 26 includes two steps 2632a, 2632b, and the ability to add one or more steps via add step element 2634. Although illustrated with two steps it should be appreciated that any number of steps may be used without departing from the spirit and scope of the present disclosure. The interface 2600 further includes a step details section 2640. The step details section 2640 may include editable sections for a step title, URL, action, description, or any other settings, operations, data, metadata, or the like in relation to a selected step (e.g., Step 1 in FIG. 26). An action input section 2650 may include a value section where an input type and variable may be associated with at least one step.

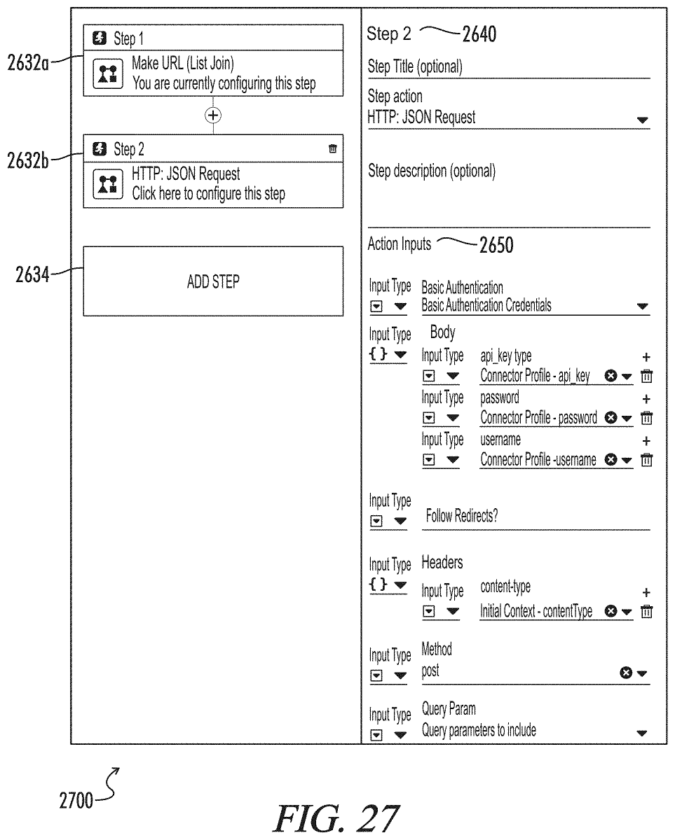

FIG. 27 illustrates an exemplary embodiment of a partial interface for a second step of the interface of FIG. 26 according to aspects of the present disclosure. The interface 2700 includes various features previously described with reference to FIG. 26 but with Step 2 selected in FIG. 27 rather than Step 1 selected as in FIG. 26.

As used herein, each workflow may be configured to run in full isolation, along with its steps, each step having its own process supervised by the workflow process and may leverage the actor model for concurrency and scalability. Secure File Transfer Protocol (SFTP) and Simple Object Access Protocol (SOAP) may be configurable via the Destinations web application. For example, users can upload Web Service Definition Language (WSDL) files that may be parsed into one or more connectors with connector actions defined by parameters included in the WSDL file. These connector actions can then be selected when building workflow steps and oat runtime may be sent to a SOAP module for communication.

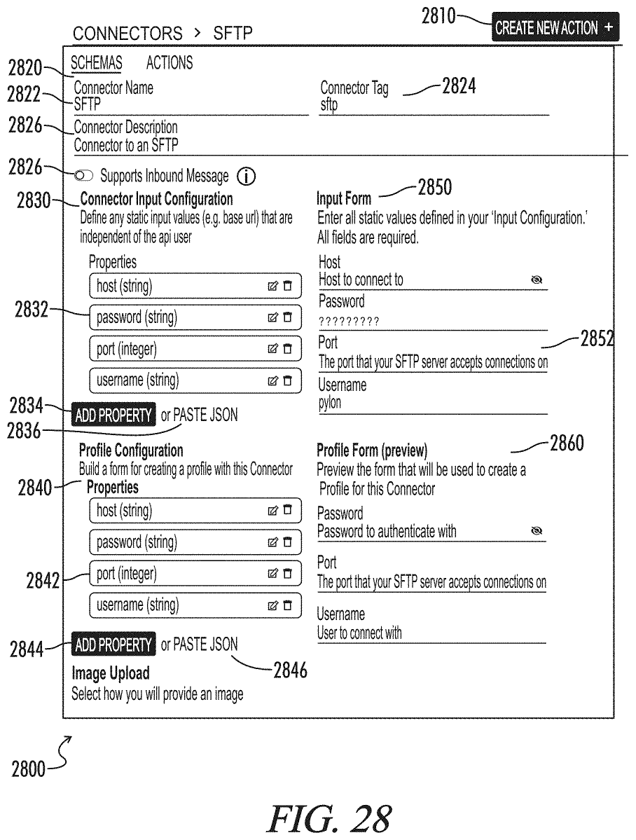

Destinations SFTP capabilities may permit users to configure SFTP-type connectors without needing to write any code, for example as illustrated by FIGS. 28-30.

FIG. 28 illustrates an exemplary embodiment of an SFTP configuration interface 2800 according to aspects of the present disclosure. The SFTP configuration interface 2800 includes a create new action element 2810. A connector section 2820 may include a connector name section 2822, a connector tag section 2824, and/or connector description and messaging section 2826, one or more of which may be created, edited, or manipulated by a user of the interface 2800.

A connector input configuration section 2830 may include one or more properties which may be edited by the user of the interface 2800 and selectively saved via an add property button 2834 which is used to save one or more properties associated with the connector. A paste JSON link 2836 may be used to import and/or export information relating to at least one of the connector and/or at least one property or configuration element associated with the connector. A profile configuration section 2840 may provide one or more properties 2842. selectively saved via an add property button 2844 which is used to save one or more properties associated with the connector. A paste JSON link 2846 may be used to import and/or export information relating to at least one of the connector and/or at least one property or configuration element associated with the connector.

An input form section 2850 may include one or more parameters 2852 associated with the connector, such as a host name, password, port, username, or other parameter. A profile form preview section 2860 may preview at least a portion of the form that will be used to create a profile for the connector.

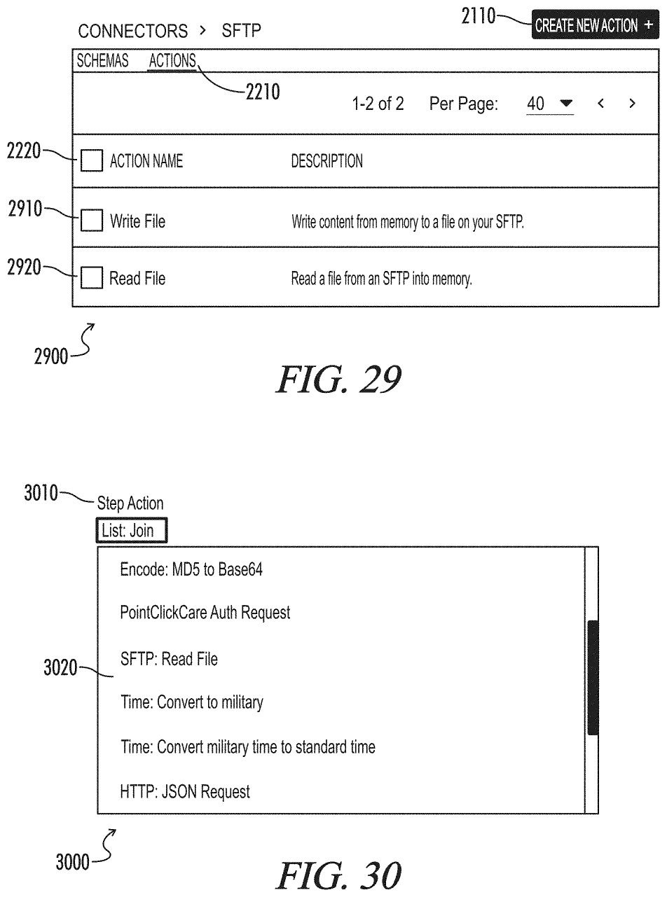

FIG. 29 illustrates an exemplary embodiment of an interface 2900 for viewing or specifying one or more actions for a connector according to aspects of the present disclosure. The interface 2900 includes the create new action element 2110, an actions selector 2210, an action information section 2220, a write file selector 2910, and a read file selector 2920. The write file selector 2910 and/or read file selector 2920 may correspond to one or more SFP operations previously created in a manner consistent with the previous disclosure.

FIG. 30 illustrates an exemplary embodiment of an interface element for selecting a step action according to aspects of the present disclosure. A drop-down list 3000 may include a step action selection section 3010. When selected, one or more selectable actions 3020 may be selected by a user. The one or more selectable actions 3020 may include at least one SFTP action defined previously as described herein. That is, actions may become available when created and may be used as a user builds his or her workflow(s).

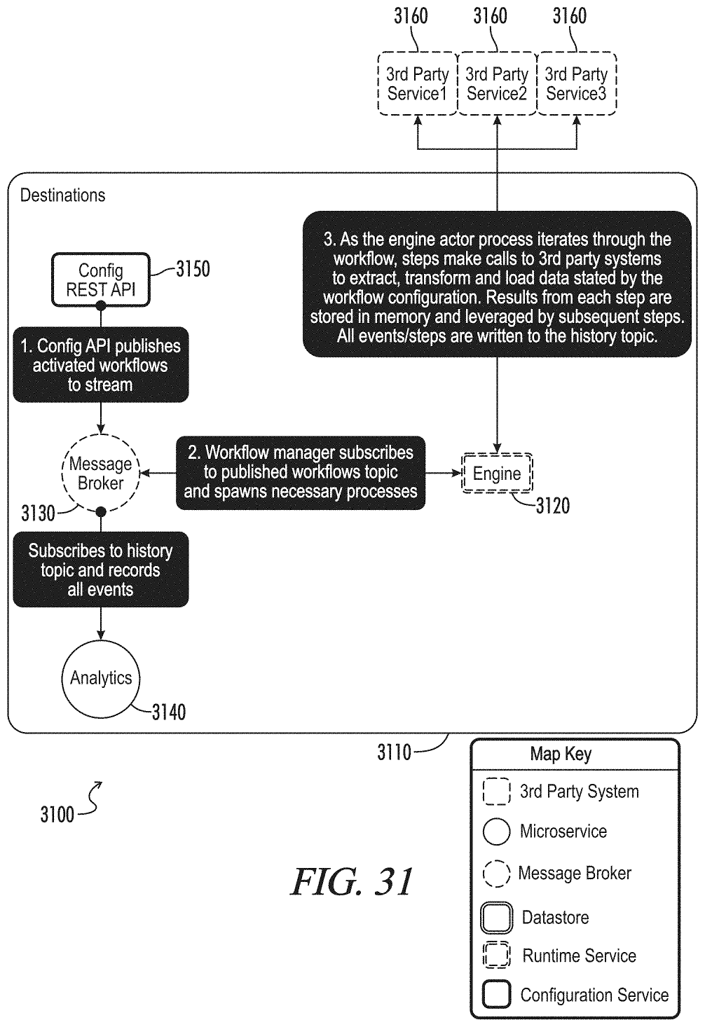

FIG. 31 illustrates an exemplary embodiment of a partial block diagram of a destinations entity 3110. The destinations entity 3110 includes an engine 3120 communicatively coupleable to a message broker 3130 (which may be configured to perform one or more services as previously described with reference to message broker 140) and/or to at least one third-party service 3160. The message broker 3130 may be coupleable to an analytics engine 3140 and/or configuration REST API 3150. In operation, the configuration REST API may publish activated workflows to a stream associated with the message broker 3130. A workflow manager of the engine 3120 may subscribe to a published. workflows topic and may spawn one or more necessary processes corresponding to a published workflow topic. As the engine actor process iterates through the workflow, steps may make calls to third-party systems 3160 to extract, transform, and load data stated by the workflow configuration. Results from each step may be stored in a memory of the engine 3120 and may be leveraged by subsequent steps. All events and/or steps may be written to a history topic. The analytics engine 3140 may subscribe to the history topic and may record all events (e.g., for later analysis and/or processing, for example using AI to predict connectors and/or connector parameters).

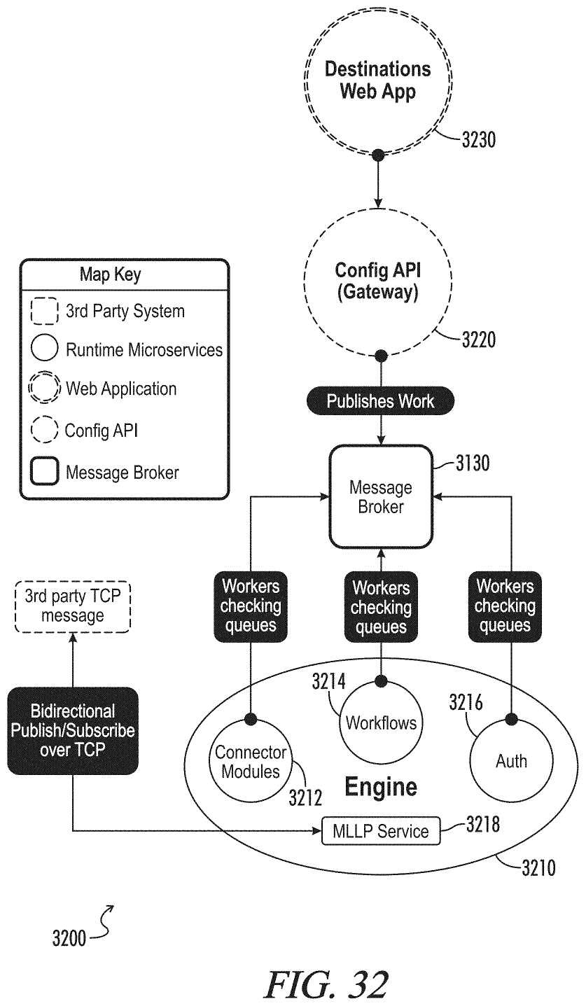

FIG. 32 illustrates an exemplary embodiment of a simplified block diagram reflecting an alternative embodiment corresponding to FIG. 3 according to aspects of the present disclosure. The system 3200 includes an engine 3200 (e.g., corresponding in various embodiments to the engine 3120 illustrated by FIG. 31). The engine 3120 may include connector modules 3212, workflows 3214, identity management module 3216, and a Minimum Lower Layer Protocol (MLLP) service which is communicatively coupleable to a third-party element via a third-party CP message using bidirectional publish/subscribe over TCP (although other protocols, data formats, and communication systems may be used without departing from the spirit and scope of the present disclosure.

At least one of the connector modules 3212, workflows 3214, and/or identity management modules 3216 may be coupleable to one or more workers checking queues, each of which is subsequently coupleable to the message broker 3130 (which may perform one or more functions previously described with reference to message broker 140. The message broker 3130 may be coupled to a configuration API (e.g., gateway) 3220. The configuration API 3220 may be configured to publish at least one work to the message broker 3130. The configuration API 3220 may be coupleable to a destinations web app 3330.

In implementations consistent with FIG. 32, a workflow may use MLLP socket traffic. In this embodiment, MLLP connectivity is moved to the engine as its own service. This provides greater capability because the system is able to treat the MLLP service as any other, thereby allowing users to configured MLLP-type connectors via the destinations user interface of the destinations web app 3330.

A user may be permitted to access and/or create information relating to one or more methods associated with the connector profile. The user may define a method for communication with a connector and select to create a new instance. The user may then provide one or more sets of information relating to instance details, add authentication details, and save such information with the connector profile. Although specific protocols, socket information, and general communications information is provided in the Map Glossary of FIG. 16, it should be appreciated that any protocol, connector, communications media, hardware or software element, or any other element may be used within the scope of the present disclosure to communicate information and/or provide connector or mapping information.

In practice, a user may create a workflow start to finish via one or more interfaces and systems described herein. To create a workflow, a user may first navigate to a workflows page. As used herein, the term workflow or workflows may refer to orchestrated containers of steps. Once on the workflows page, a user may select a “Create” option and may provide a name for a new workflow. The user may then select a “Create” option for a new step of the new workflow. The user is enabled to create step information, for example by selecting a connector to use and/or communication representation. A user may additionally or alternatively create step information by filtering patient matching using EMPI services, translation, and/or transformation rules. Results from one step may be optionally provided to a subsequent step in a workflow. The process is continued until a desired workflow or sequence is complete.

Apparatuses, systems, and methods consistent with the present disclosure may enable a user to create and/or modify one or more connectors. The user may begin by logging into an interface or standalone application. The user may navigate to a connectors page (e.g., using a build communication link to desired system interface). The user may select a “Create” option on the connector page to create a new connector. The user may add one or more communication methods after creating the connector. For each communication method the user may describe details necessary for communication. Additionally or alternatively, at least one set of predetermined or dynamically determined information may be used to prepopulate and/or suggest one or more details. A user may be capable of automating at least a portion of connector creation by parsing a swagger document or other document type, for example. A user may further automate at least a portion of connector creation by supplying or uploading a sample of data and by filling out the gaps via a web application or interface. A user is further capable of automating at least a portion of the connector creation process using a web browser extension which is capable of obtaining one or more actions associated with a vendor system and recording the action(s) and/or information associated with at least one action, such recorded action(s) and/or information being stored by the system disclosed herein and/or a system communicatively coupleable to the system. Additionally or alternatively, a user may use a connector wizard to enter information about a device which they are attempting to communicate with. Finally, a user may select a “Verify” option that may be configured to attempt to authenticate to ensure that the connector details are correct.

Systems consistent with the present disclosure may use a collection of translation libraries to assist in formatting conversions. After the data has been identified, authenticated, and authorized, the API gateway may call upon the connector API to request the formatting information. If formatting is needed, the API gateway may call upon the necessary library for translation and pipes the data through. Both the raw and translated sets of data may be sent to an analytics module as well as the workflows module for processing. A plurality of translation protocols, formats, and methods may be used consistent with the present disclosure without departing from the spirit and scope of the disclosure.

Predictive workflow and step field mapping may be implemented in various embodiments. Using analytics field mapping model, implementations consistent with the present disclosure may be capable of processing a handful of key inputs that determine high level details of the connector profile to generate a high-fidelity prediction of the mapping. Similarly, one or more aspects of a connector profile may be predicted. For predictive connector profile operations, multiple data science approaches may be used that allow at least a portion of the connector build process automated. A service may ingest a sample data set and process it against a current analytics engine's connector profile model to predict and generate the required connector for communication with the desired system. Additionally or alternatively, a browser extension may be implemented that records user interaction with the third-party system and sends such recorded data back to the analytics engine for processing, prediction, and connector generation.

The workflows module described herein may rely heavily on JSON formatted objects in various embodiments. These objects may contain data, instructions, and logic rules. A mapping may be useable by the gateway to process and route communication(s).

Webhooks/Incoming Messages

For Workflows that begin with a Webhook, the entire record may be retrieved automatically before passing it into a Workflow Handler (e.g., workflows module), so there is no need to create an extra step to go and query that record.

Similarly for incoming message-initiated workflows, the entire message itself may be parsed and available as the results of the first step.

Mapping Data

As seen in the example below, values are typically mapped from previous steps. These values may be used directly for the end-goal step (such as create/update) or may be used in conjunction with an ID retrieved in a previous step to get another record that contains the required data for the end-goal step. Either way, the ‘source’ key may be used.

For the field ‘ssn’, data may be pulled from the first step (index of ‘0’ is used to match the standard developer index), where the field name is ‘SSN_c’. If there is a non-null value available, then it may be used for the current step as ‘ssn’=>‘111-11-1111’, for example.

With the use of multiple sources and a separator key, the ‘FirstName_c’ and ‘LastName_c’ from the first step may be combined with a space in between, so that the end result would be ‘Name’=>‘John Doe’.

Special Mapping Requirements for Some Methods

Some methods might require certain fields to be in the map in order to successfully complete the request. This is because these methods might require a record ID to complete the request (i.e., it cannot just be sent as one big string of data). Instead, it might be advantageous to tell the service which ID is planned to be used as the lookup, etc.

Update, getById, and getAttachment methods may require an ‘id’ key, where that can be exactly a lowercase ‘id’, or it can also be the name of the record ID that the service specified, such as ‘recordId’. This value may be required in the map to properly perform these methods in various embodiments:

The “update or insert” (also referred to as an upsert) may require an external ID field name, as well as a pointer to what that field name should be. An ‘external_id_field_name’ value may be used to tell Workflows we will be using the value mapped for Ping_External_ID_c as the external ID argument.

Conditional Mapping

There are scenarios where data might want or need to be mapped based at least in part upon on certain conditions, such as “If the City field from step 2=‘Chicago’, then mark the ‘Zone’ field as 1, else if it's ‘Naperville’, then map the value from ‘Zone’ from the first step. Otherwise, mark it as 2”.

Conditional logic may be activated if the output mapping has a ‘logic’ key. The logic key may contain an array of conditions that act as if/else if statements. Each one of those conditions may contain one or many ‘and’ conditions as well as ‘or’ conditions. All ‘and’ conditions' may pass in order to use the condition's ‘passes’ values. However, only one of the ‘or’ condition must pass to use the success value in the ‘passes’ key. The ‘prioritylndex’ key may be added to map that particular field first, in case other fields within the same step depend on the outcome of that particular mapped field, i.e., “If the field Gender c was already mapped in this same step as x, then evaluate logic y”. The value in ‘passes’ can be a static value or mapped from the result history (i.e. from a previous step).

Conditional Step Execution

One or more Steps may be skipped during operation depending at least in part upon the value of previous result fields. For example, let us say for Step 2 it is necessary to query a related Insurance object. However, it might only be required that the source field on the first step (let us say it is “Related_Insurance_ID_c”) is NOT null, i.e. it has a value. To accomplish this, the ‘conditions’ setting may be used, where the operator may be: ‘>’, ‘<’, ‘>=’, ‘<=’, ‘=’, ‘!’ (NOT equals), ‘like’ or ‘contains’ (string contains), ‘!like’ or ‘!contains’ (string doesn't contain). A field from a previous step may be compared to a static value, and if it is false, the step will not run, but the workflow will continue with any future steps.

Multiple conditions may be added, and are evaluated as ‘and’ statements, i.e., ALL must pass. If the ‘logic_type’ key is passed with value ‘or’, then that condition is evaluated as an or statement, meaning ANY of these ‘or’ conditions passing will run the step, despite how many other conditions might fail.

Transformation

After a value is mapped, transformations may be applied. Transformations may be located in the same JSON level as the source and default.

Date transformations of any kind might require the ‘toDate’ key, where the value is the string format we would like to output. In the example above, this would take the value from the first step DOB_c field and attempt to parse it as a date (using the Carbon library), and further output it into the correct format. Sometimes the ‘fromDateFormat’ string is necessary to declare the expected format from that step's output. For example, this particular service is sending the date as 20190101030355, in which case we want to explicitly tell Carbon that this string of numbers is actually in the format of ‘YmdHis’. Finally, the ‘toTimezone’ setting is applied to change it into the customer's time zone.

Prepend simply adds a value to the beginning of the mapped value. Output above would be “Name”=>“Mr Smith”.

Substring returns the substring of the output value, which is sometimes useful for trimming data. This feature follows the same standard substring index logic.

Translate will take exact matches and translate them into another value, e.g. if the value of

‘Patient_Type’ was ‘A’ then go ahead and map as “Type_c”=>“Inpatient”.

Merging Results

This setting is used for workflows that require getting multiple attachments from the object. A ‘transform’ setting may be used to merge the attachment results for the workflow to function properly:

Filtering Records

A very common requirement is to filter the record results. This allows us to only pass on records that meet necessary requirements. This is especially useful for polls where the first step may be listByDateRange, where created records may be checked for a given date range. This can obviously produce many results, so sometimes it might be necessary to reduce that number to meet criteria.

After all results are obtained, it is possible to limit to the records that have been updated in the last 65 minutes (type ‘date’ is required for ANY date filters), and where the discharge date is not null, i.e., it has a value.

Apparatuses, systems, and methods consistent with the present disclosure may receive numerous benefits over previous systems. For example, the speed of general integration is radically increased relative to existing systems based at least in part upon the lack of coding knowledge or other technical knowledge required by a user to create both workflows and connectors. Systems consistent with the present disclosure may be intuitive in that the core application and data-driven automation tools permit prediction for an entire connector, including data mapping, data translation, and connector configuration.

The approach provided by the present disclosure provides added integration flexibility in multiple ways. First, the connectors microservice permits creation of virtually any type of connector used in healthcare over a variety of communication methods. Using a node-based network layer permits microservices to be dropped inside data centers and permits relay of messages back to the engine in real-time via a web socket secure protocol, thereby allowing facilities without API communication to utilize the platform.

To facilitate the understanding of the embodiments described herein, a number of terms are defined below. The terms defined herein have meanings as commonly understood by a person of ordinary skill in the areas relevant to the present invention. Terms such as “a,” “an,” and “the” are not intended to refer to only a singular entity, but rather include the general class of which a specific example may be used for illustration. The terminology herein is used to describe specific embodiments of the invention, but their usage does not delimit the invention, except as set forth in the claims. The phrase “in one embodiment,” as used herein does not necessarily refer to the same embodiment, although it may.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The previous detailed description has been provided for the purposes of illustration and description. Thus, although there have been described particular embodiments of new and useful inventions, it is not intended that such references be construed as limitations upon the scope of this disclosure except as set forth in the following claims.

What is claimed is:

1. A method for providing healthcare integrations, comprising:
receiving a request to generate a connector;
identifying a source and a destination for the connector;
selecting at least one action associated with the connector;
defining a parameter associated with the at least one action; and
generating the connector based at least in part upon the identified source and destination, and the selected at least one action, the defined parameter.

2. The method of claim 1, further comprising:
generating an interface for a user to provide at least one of the identified source and destination, the selected at least one action, or the defined parameter.

3. The method of claim 1, further comprising:
implementing an artificial intelligence (AI) component; and
predicting at least one connector setting based at least in part using the AI component.

4. The method of claim 3, further comprising:
storing historical connector information relating to at least one connector, wherein the predicting the at least one connector setting is based at least in part upon the historical connector information.

5. The method of claim 1, further comprising:
translating at least a portion of the at least one action based upon a defined data model.

6. The method of claim 1, further comprising:
providing a gateway to coordinate communications with at least one third-party Application Programming Interface (API) forming at least a part of the connector.

7. An apparatus for providing healthcare integrations, comprising:
a core gateway configured to communicate with at least one third-party Application Programming Interface (API);
a storage configured to store connector profile information;
a server configured to generate an interface associated with at least one connector, the interface configured to permit a user to generate at least one connector;
an analysis engine configured to interpret at least one communication associated with a connector and configured to store a representation of the interpreted at least one communication associated with the connector; and
a connector generator, the connector generator configured to generate a connector based at least in part upon.

8. The apparatus of claim 7, wherein the analysis engine includes an artificial intelligence (AI) component configured to analyze at least a portion of the connector profile information stored at the storage and to predict information regarding at least one connector.

9. The apparatus of claim 7, further comprising:
a connector generator configured to generate a connector based at least in part upon an identified source and destination, at least one action, or a defined parameter associated with the connector.

10. A system for providing healthcare integrations, comprising:
a network;
a server communicatively coupleable to the network, the server configured to generate an interface accessible via the network;
a plurality of third-party service Application Programming Interfaces (APIs) coupleable to the network; and
a user device coupleable to the network, the user device configured to access the interface generated by the server to generate a connector, the connector configured to include at least one of the plurality of third-party service APIs.

11. The system of claim 10, further comprising:
an artificial intelligence (AI) component configured to predict at least one connector setting based at least in part using the AI component.

12. The system of claim 11, further comprising:
a storage configured to store historical connector information relating to at least one connector, wherein the AI component predicting the at least one connector setting is based at least in part upon the historical connector information.

13. The system of claim 12, wherein the system is configured to translate at least a portion of information relating to the at least one connector setting based upon a defined data model.

14. The system of claim 13, further comprising a core gateway configured to communicate with at least one of the plurality of third-party service APIs, the core gateway configured to coordinate the translation of the at least a portion of information relating to the at least one connector setting.

15. The system of claim 10, further comprising a core gateway configured to communicate with at least one of the plurality of third-party service APIs.
